(19) United States
(12) Patent Application Publication
(10) Pub. No.: US 2024/0117441 A1
(43) Pub. Date: Apr. 11, 2024

(54) TUMOR MICROENVIRONMENT BY LIQUID BIOPSY

(71) Applicant: LiquidCell Dx Inc., Chesterfield, MO (US)

(72) Inventors: Aadel Chaudhuri, Chesterfield, MO (US); Aaron Newman, Chesterfield, MO (US)

(21) Appl. No.: 18/484,726

(22) Filed: Oct. 11, 2023

Related U.S. Application Data
(60) Provisional application No. 63/482,314, filed on Jan. 31, 2023.
(60) Provisional application No. 63/415,010, filed on Oct. 11, 2022.

(51) Int. Cl. C12Q 1/6886

(52) U.S. Cl. CPC C12Q 1/6886; G01N 2800/52; G01N 2800/7028

(57) ABSTRACT

The invention provides methods to noninvasively profile the tumor or the tumor microenvironment using a body fluid sample such as a blood draw. Methods of the disclosure use a liquid biopsy analysis to profile immune cell abundance and diversity in the tumor microenvironment and to predict a patient's response to therapy, particularly to immunotherapy. Specifically, methods of the disclosure are useful to predict immunotherapy toxicity (and treatment toxicity more generally) from cell-free DNA methylation cell state analysis.

### TECHNICAL FIELD

The disclosure relates to noninvasive profiling of the tumor microenvironment and predicting immunotherapy response & toxicity from markers in cell-free DNA.

### BACKGROUND

A tumor is a complex ecosystem that includes cancer cells among a variety of other cells, some of which help the tumor to grow, while some try to kill it and others serve as bystanders. Tumors may also include blood vessels and extracellular matrix. The environment in which cancer cells reside is known as the tumor microenvironment (TME). See, Anderson, 2020, The tumor microenvironment, Current Biol 30(16):R921-R925, incorporated by reference.

Leukocytes (immune cells) are an important part of the TME and are also found circulating in blood. Leukocytes that infiltrate the tumor are referred to as tumor infiltrating leukocytes (TILs) while those circulating in blood are peripheral blood leukocytes (PBLs). See, Chen, 2013, Oncology meets immunology: the cancer-immunity cycle, Immunity 39(1):1-10, incorporated by reference. Some important examples of leukocyte types are cytotoxic T cell, Tregs, B cells, NK cells, and Neutrophils.

The evolution of a tumor largely depends on the TME, particularly TILs. For example, cytotoxic T cells can be helpful to kill cancer cells whereas Tregs can help the tumor to grow. The role of TILs within the TME is variable, and those cells can potentially be tumor cell killers or promoters of tumor cell growth or can even change roles depending on conditions.

### SUMMARY

The invention provides methods to noninvasively profile a tissue microenvironment using a body fluid sample. Methods of the invention are useful to analyze and detect aberrant tissue microenvironments (ATMs), such as those caused by tumors (tumor microenvironments), inflammatory conditions (inflammatory microenvironment), tissue transplants (transplant microenvironment), and various pathogens (infectious microenvironments). The invention provides a noninvasive computational end-to-end framework for profiling an ATM using liquid biopsy samples (LiquidTME) and sequencing relevant nucleic acids, in particular, cell-free DNA (cfDNA). In preferred aspects, LiquidTME methods of the invention analyze and profile ATMs, including those of tumors, using cell-free DNA methylation and cell state analysis in nucleic acid obtained from a liquid biopsy sample. Methods of the invention provide a cellular profile in a particular microenvironment.

LiquidTME methods disclosed herein provide robust and sensitive profiles of distributed tissue microenvironments in a subject using only a liquid biopsy sample. As described herein, analyses and profiles of various microenvironments produced using methods of the invention have been validated using data generated from different assays. Methods disclosed herein have been validated and are more sensitive than many conventional first-line disease analysis techniques. For example, methods of the invention provide early detection of a TME that has or is undergoing a transition from a benign state to a malignant state. The presently-disclosed methods detect a malignant TME, using predictions generated from liquid biopsy samples, even in patients with low tumor burden. Thus, methods of the invention provide an advance in the standard of care for assessing cancer.

Similarly, methods of the disclosure use liquid biopsy analysis to profile the immune cell repertoire e.g., immune cell abundance and diversity, in a microenvironment to predict a patient's response to a therapy. In certain aspects, methods of the disclosure are used to predict immunotherapy toxicity (and treatment toxicity more generally) and/or response durability based on cell-free DNA methylation patterns and cell state analysis. For many diseases, immunotherapies are the only effective treatment for some individuals, while in others it produces no improvement or even deleterious effects. Consequently, immunotherapies are often a secondary treatment option, only provided after first-line treatments have proven unsuccessful.

For example, it is understood that some patients, particularly cancer patients, experience a category of toxic effects in response to immunotherapy known as immune-related adverse events (irAE). It is also understood that not all patients will respond positively (regardless of irAE) to immunotherapy. Methods of the invention provide a profile of the cellular makeup of an ATM that is predictive of immunotherapy toxicity and patient response. The analysis may be made to generate the predictive profile before a patient is given a pre-immunotherapy, such as an immune checkpoint inhibitor.

A provided profile may include, for example, a composite model that gives measures of both activated CD4 T effector memory (TEM) cell abundance and T cell receptor (TCR) diversity. Data show that CD4 TEM abundance or TCR diversity can be predictive of immunotherapy response or toxicity and the present invention provides measures of CD4 TEM abundance and TCR diversity by analyzing cell-free DNA (cfDNA) from a blood or plasma sample obtained non-invasively.

Specifically, embodiments of the invention include identifying epigenetic modifications in cfDNA and measuring TEM abundance or TCR diversity from patterns in the epigenetic modifications. For example, using a technique, such as bisulfite sequencing or enzymatic methyl-seq (EM-seq), methods of the invention provide data that describe patterns or locations of methylated DNA on nucleic acids from an ATM. Deconvolution and assembly software identifies which genes have levels of methylation (e.g., promotor methylation) in the ATM. Those results may be added to, or compared to, a liquid biopsy atlas that provides a mapping of cfDNA sequence patterns to cell states, disease states, or physiological states. After deconvolution and assembly (which identifies patterns of epigenetic modifications on a gene-specific level), patterns of epigenetic gene modification, or sets of modified genes, can be looked up in the atlas to quantify or identify cell or tissue types or states and to predict or monitor risk or status of a wide range of physiologic states, disorders, infections and diseases.

The read assembly and atlas lookup identifies cells or specific cellular state(s) (e.g., transcriptional states) that are or have been present in the ATM of the patient. Identifying that the ATM has cells of a set of certain cell states is strongly predictive of disease progression and/or irAE (e.g., toxicity) in immunotherapies, such as immune checkpoint inhibitors; and is also predictive of patient response, i.e., of whether that patient will respond favorably to immunotherapy. The read assembly and atlas may also provide a measure of T cell abundance and diversity (e.g., TEM abundance and TCR diversity), and that provides a profile of TILs and the immune repertoire in an ATM.

Methods of the disclosure are useful to granularly profile peripheral blood cell states and/or quantify activated immune cells from cell-free DNA. Such methods provide for the pre- and early on-treatment prediction of immunotherapy toxicity (i.e., immune-related adverse events or irAE). Moreover, methods of the disclosure are useful to concurrently predict both treatment response and toxicity using the same assay. Additionally, methods of the disclosure are useful to concurrently quantify a wide range of cell states that comprehensively represent human health and disease, essentially providing an atlas (i.e., by measuring multiple cell, tissue and microbial types/states that includes what has been sequenced or what is present in public/published methylation datasets), to predict and monitor risk for a wide range of physiologic states, disorders, infections and diseases. Thus, the invention provides methods for cfDNA methylation analysis of cells/tissue states to predict treatment response and toxicity.

Methods of the disclosure are useful for noninvasive early/pre-treatment prediction of severe immune-relative adverse events by cell-free DNA analysis; concurrent prediction of both severe immune-related adverse events and immunotherapy response by early/pre-treatment cell-free DNA analysis; providing a liquid biopsy atlas of human health and disease with the ability to quantify multiple cell/tissue/microbial types/states; and predicting and monitoring risk/status of a wide range of physiologic states, disorders, infections and diseases.

In certain embodiments, the invention provides methods for deconvolving sequence reads and read counting applicable to cell-free DNA methylation data, where the methods include (i) identifying CpG sites on a per-fragment level in cell-free DNA; (ii) comparing, per CpG site per fragment, methylation levels to ground-truth reference table of known cell/tissue states; and (iii) counting or collating across CpG sites in this way per fragment to assign the cell-free DNA fragment to a cell state within the reference table. Preferably, methods include (iv) continuing the counting of cell-free DNA fragments until substantially all fragments are analyzed for assignment to a cell state and/or (v) determining, from the cell state assignments, the cell state composition of the cell-free DNA mixture (i.e., from the liquid biopsy sample). The CpG sites per fragment may be provided as inputs to a machine learning algorithm that performs the assignments of fragments to cell states. The cell state composition may be provided as a report of the ATM infiltrating immune cells, such as leukocytes of the tumor microenvironment. Thus, the disclosure provides liquid biopsy measures of the ATM. Methods of the disclosure provide high-resolution methylation cell state analysis of plasma cell-free DNA. Methods herein are useful to quantify an arbitrarily high number (e.g., >20 or >50 or more) distinct cell states in blood plasma. Methods herein provide the ability to concurrently predict immunotherapy response and toxicity from the same exact plasma sample and sequencing result, and to do so pre-treatment. Methods of the disclosure have important clinical implications for pre-treatment/early immunotherapy response and toxicity prediction.

In certain aspects, the invention provides a method of predicting an effect of therapy. The method includes identifying epigenetic modifications in nucleic acid from a sample, measuring T cell abundance and/or diversity from patterns in the epigenetic modifications, and predicting a response to therapy based on the T cell abundance and/or diversity. The measured T cell diversity may be a T cell receptor (TCR) diversity of a patient's immune repertoire. The method may include predicting a risk of an adverse event in response to, or predicting responsiveness to, immunotherapy when the TCR diversity is beneath a predetermined threshold.

In preferred embodiments, the sample is a blood or plasma sample or urine sample or other biofluid sample obtained by liquid biopsy. Preferably the nucleic acid is cell-free DNA. The identified epigenetic modifications may include promoter methylation. The method may include querying a cell state atlas for the patterns in the epigenetic modifications. That is, a set of gene promotors methylated in the sample can be looked up in the atlas to identify a cell state associated with that set of methylated gene promoters. The method may include providing a profile describing cell states of a plurality of cells in a tumor microenvironment in a patient. Such a profile may include, for example, transcriptional states of cells such as a list of cells present and transcription levels of certain genes in those cells. In some embodiments, the method includes providing a profile of tumor-infiltrating leukocytes in a tumor microenvironment based on the patterns in the epigenetic modifications. Preferably, the method includes inferring more granular tumor-infiltrating leukocyte and other tumor microenvironmental states within this cell-free DNA TME/ATM compartment (i.e. inference of the tumor microenvironment composition from cell-free DNA analysis).

In certain aspects, the present invention provides methods of predicting a disease outcome and/or to risk stratify a patient. An exemplary method includes identifying epigenetic modifications in sequence data that includes methylation status of nucleobases from cell-free nucleic acids from a liquid biopsy sample obtained from a subject; identifying an aberrant tissue microenvironment and assigning a tissue of origin to the cell-free nucleic acids based on the patterns in the identified epigenetic modifications; determining cell states in the aberrant tissue microenvironment using patterns epigenetic modifications; and predicting the outcome of a disease in the subject based on the cell states in the aberrant tissue microenvironment.

In certain aspects, the aberrant tissue microenvironment is selected from a tumor microenvironment (TME), an inflammatory microenvironment (IME), a tissue transplant microenvironment (TTME), and a pathogen microenvironment (PME). In some embodiments, the circulating cell-free nucleic acids are from microenvironment infiltrating lymphocytes. In certain embodiments, the disease is cancer and the aberrant tumor microenvironment is a TME.

In certain methods, predicting the outcome of cancer in the subject includes one or more of diagnosing the subject with a cancer, predicting remission, predicting recurrence, assessing minimal residual disease, predicting response to an immunotherapy, and predicting immunotherapy toxicity. In general, method of the invention allow risk stratification in patients with locally advance or metastatic cancer, and is useful to distinguish patients with molecularly-low risk disease from those with molecularly-high risk disease. This type of stratification is applicable across cancers and is an aid to enable and strengthen personalized treatment.

In certain aspects, methods of the invention are able to detect a tumor microenvironment, and thus cancer, in a subject with a low measured tumor burden or in the cancer screening setting (in a patient without known cancer diagnosis). Predicting the outcome of cancer in the subject using methods of the invention may include determining that the cell states in the TME have surpassed the pre-malignant threshold. This may be based on granular TME cell state compositions and/or T cell abundance and/or diversity from patterns in the epigenetic modifications. The measured T cell diversity may be the T cell receptor (TCR) diversity of a patient's immune repertoire. Certain methods of the invention may include a step of inferring an abundance of T cell effector memory cells from the patterns.

In certain aspects, methods of the invention include predicting a risk of an adverse event in response to immunotherapy wherein the measured TCR diversity and/or inferred T cell effector memory cell abundance is above a predetermined threshold. Methods may further include predicting a positive outcome in response to an immunotherapy wherein the measured cell-free DNA-derived TME profile or abundance of certain cell states within the TME or abundance of total tumor-infiltrating leukocyte-derived cell-free DNA is above a predetermined threshold.

In preferred methods, the step of determining cell states includes sequencing nucleic acids from purified versions of those cell states to produce the sequence data that include methylated bases; mapping the sequence data to a reference to identify promotors of a plurality of genes; and identifying the cell states based on sets of the genes having hypomethylated promotors.

Methods of the invention may include a step of providing a profile of tumor-infiltrating leukocytes in a tumor microenvironment based on the patterns in the epigenetic modifications.

In certain methods, the cell-free nucleic acids include nucleic acids from non-tumor cells and the step of determining cell states in the aberrant tissue microenvironment includes generating a profile of the TME. The non-tumor cells may include one or more of stromal cells, immune cells, and/or cells from the tumor margin. The profile of the TME may include one or more epigenetic pattern correlated with tumor progression and/or tumor regression.

Methods of the invention may predict toxicity of an immunotherapy based on a level of epigenetic modification of promotors in genes involved in a predetermined T cell transcriptional state and/or other immune cell states; and predicting immunotherapy response based on a level of promotor methylation in the genes corresponding to a predetermined tumor microenvironmental cellular community. The predetermined T cell transcriptional state may be specific to CD4 T cells. Methods of the invention may also predict toxicity of an immunotherapy based on a level of epigenetic modification of promotors in genes involved in the expression of one or more cytokine; and predicting immunotherapy response based on a level of promotor methylation in the genes in a predetermined tumor ecotype.

In certain aspects, the epigenetic modification predictive of immunotherapy toxicity and is selected from one or more of: promoter hypomethylation of interleukin-10; promoter hypermethylation of interleukin-6; and promoter hypermethylation of interleukin-7.

In preferred embodiments, the invention comprises selecting fragments of a preferred size for TME analysis. For example, selecting for cfDNA fragments having a size consistent with fragment sizes produced by tumors allows the subsequent aneuploidy or other analysis to focus on tumor-derived DNA. Thus, liquid TME analysis can be based on a fragmentomic screen that focuses on fragments known to be associated with a tumor, including specific end motifs in cell-free DNA fragments. In one embodiment, copy number of cfDNA fragments is used as a proxy for aneuploidy. In another embodiment, aneuploidy analysis is combined with other diagnostic criteria, such as tumor mutational burden, to stratify patients with respect to immunotherapy response. In this way, a blood sample can be deconvolved into a matrix that contains fragment size, copy number, mutational burden, and/or epigenetic signatures in order to provide fine resolution on prospective immunotherapies and potential disease progression and outcome.

Methods of the invention also utilize an LTME signal from a tumor to determine aneuploidy and to predict disease outcome. In general, aneuploidy is predictive of response to immunotherapy in most patients. According to the invention copy number determinations in an LTME sample are used as a surrogate for aneuploidy. Highly-altered copy number is predictive of high aneuploidy and low copy number is predictive of low aneuploidy. Thus, LTME signal is useful to predict response to immunotherapy as a proxy for aneuploidy.

In another aspect, LTME samples are used to select for nucleic acid fragment size as a proxy for aneuploidy. For example, tumor-derived cfDNA in a liquid biopsy sample is typically shorter than cfDNA from non-tumor cells. Thus, cfDNA is size-selected and used in LTME as a filter for aneuploidy and resulting stratification of patients as to risk of severe disease and/or response to immunotherapy. In general, LTME analysis based on cfDNA fragmentomics is a good predictor of immunotherapy success (e.g., efficacy and likelihood of adverse events).

An alternative embodiment is a hierarchical strategy to address multicollinearity. Broadly, in the first step, cell types are grouped into broader classes (e.g., all T cells, all B cells) based on a measure of similarity between cell type methylation profiles (e.g., promoter-level methylation across the genome, cell type-enriched CpGs detected by feature selection, etc.). In this way, cell types with a given class will exhibit multicollinearity with each other (e.g., CD4 effector vs central memory T cells) whereas cell types between classes will not (e.g., CD4 effector memory T cells vs. naive B cells).

In the second step, read counting is used to distribute reads/fragments to each class with high accuracy. This is possible because each class is easily separated by highly specific CpG profiles. The reads assigned to each class are used to generate methylation profiles that are class-specific.

In the third step, cell type CpGs within a given class are used to deconvolve the bulk CpG mixture of each class (i.e., the one derived from class-specific reads). This can be accomplished by any number of statistical learning methods that regularize the result to gracefully address multicollinearity (e.g., gradient boosted decision trees [XGBoost], non-negative least squares regression with L2-norm regularization, deep learning).

In the last step, the cell type-specific fractions within each class are combined by weighting them by the global fractional abundance of each class (e.g., weight each T cell subset by the fraction of total T cells in the mixture; repeat for all cell types).

Additional examples and advantages of the present invention are provided below in the detailed description thereof.

### BRIEF DESCRIPTION OF THE DRAWINGS

FIG. 1 shows a workflow of methods of the invention.

FIG. 2 gives results showing the performance of Read-Counting.

FIG. 3 shows higher CpG per fragment results in a lower false positive rate (FPR).

FIG. 4 shows a Limit of detection analysis.

FIG. 5 graphs the number of available fragments decreases as the CpG cutoff is raised.

FIG. 6 shows FPR with cellular fraction for different numbers of CpG per fragment.

FIG. 7 gives a workflow of signature matrix generation.

FIG. 8 shows results from tumor microenvironment-based deep deconvolution.

FIG. 9 shows a signature for CD8 TIL.

FIG. 10 shows a correlation of tumor microenvironment-estimated TIL content.

FIG. 11 shows liquid biopsy to predict immunotherapy response and toxicity.

FIG. 12 is a box plot and ROC plot showing prediction of patients' response.

FIG. 13 shows the box plot and ROC plot for toxicity prediction.

FIG. 14 shows a tumor microenvironment profile.

FIG. 15 shows relative abundance of 20 cell states.

FIG. 16 shows cell state abundances (scRNA-seq) versus future irAE status.

FIG. 17 shows TCR diversity by scV(D)J-seq.

FIG. 18 shows CD4 memory T cell levels associated with irAE.

FIG. 19 shows a composite model predictive of irAE grade.

FIG. 20 shows time to severe irAE in combination ICI patients.

FIG. 21 shows TCR clonal dynamics.

FIG. 22 shows CD4 TEM levels measured by CyTOF.

FIG. 23 shows CD4 TEM expression profile.

FIG. 24 shows Tph levels measured by CyTOF.

FIG. 25 shows a pretreatment cell-free DNA analysis.

FIG. 26 shows activated CD4 TEM score and CE9 score.

FIG. 27 shows results of methods herein.

FIG. 28 shows that pretreatment CD4 Tph cells and irAE risk.

FIG. 29 shows method steps.

FIG. 30 is a schematic of the proposed project.

FIG. 31 compares TCR clonotypes to a database of CDR3 sequences.

### DETAILED DESCRIPTION

The invention provides methods to noninvasively profile a tissue microenvironment using a body fluid sample. Methods of the invention are used to analyze and detect aberrant tissue microenvironments, such as those caused by tumors (tumor microenvironments), inflammatory conditions (inflammatory microenvironment), tissue transplants (transplant microenvironment) and various pathogens (infectious microenvironments). The invention provides a noninvasive computational end-to-end framework for profiling an aberrant tissue microenvironment (ATM) using liquid biopsy samples (LiquidTME) and sequencing relevant nucleic acids, in particular, cell-free DNA (cfDNA). Methods of the invention are useful to estimate closely related cell types and states including cell states related to aberrant tissue microenvironment (e.g., tumor microenvironment) profiles from blood plasma derived cell-free DNA and apply it in different clinical settings. The disclosure provides a new platform for noninvasive profiling of infiltrating immune cells from aberrant microenvironments in patients and specifically for profiling and decoding infiltrating immune cell-derived methylation signatures identified from plasma-derived cell-free DNA molecules.

In preferred aspects, the LiquidTME methods of the invention analyze and profile tissue microenvironments using a liquid biopsy sample. Using only a sample from a liquid biopsy, methods of the invention may profile the cellular makeup of an aberrant tissue microenvironment, such as a tumor microenvironment (TME).

Methods of the invention are useful for predicting treatment responses and side effects of certain therapies, particularly immunotherapies, for patients, such as those with cancer. In certain aspects, the invention uses methylation-based cell-free DNA analysis to detect circulating DNA from a particular aberrant microenvironment and predict its tissue of origin. Methods of the invention may also include detecting and/or quantifying ATM infiltrating immune cells (e.g., tumor infiltrating leukocytes in a TME) in a sample. The present invention uses the insight that the methylation profiles of certain cells, particularly immune cells, from a particular ATM differ from those of normal cells, unassociated with the microenvironment. Methods of the invention also include method to deconvolve methylation bulk mixtures into purified cell states. Methods of the invention may use, and their results may contribute to, a database or atlas of cell state-specific methylation profiles.

To successfully ameliorate certain conditions, immune cells, such as cytotoxic T cells (CTLs), need to migrate to an aberrant tissue microenvironment to destroy any infected or diseased cells. For example, in cancer, CTLs need to migrate to the tumor microenvironment (as tumor infiltrating lymphocytes [TIL]) and be capable of destroying the cancer cells. Studies show that TILs often have limited capability in killing the cancer cells due to being in a dampened state of activity. Similar states are known to be associated with other conditions, such as inflammatory conditions or diseases caused by pathogens. This dampened state is known as exhaustion. Exhausted T cells express inhibitory receptors to which cancer cells bind corresponding ligands to avoid attack. Consequently, despite there being TILs within the TME (or other relevant microenvironment), diseased cells can escape immune-mediated killing, thus facilitating their spread, growth, and ultimately patient death. See Wherry, 2015, Molecular and cellular insights into t cell exhaustion, Nature Rev Immunol 15(8):486-499, incorporated by reference.

The present invention provides a noninvasive liquid biopsy approach to profile the cellular composition of such aberrant tissue microenvironments. Specifically, methods of the invention are useful to estimate closely related cell types and states including cell states related to ATM profiles from blood plasma derived cell-free DNA, which finds clear utility and applicability in various clinical settings. The present invention provides methods for detecting, analyzing, and predicting the outcome of treatments targeting such microenvironments. In particular, methods of the invention may be used to predict a durable response and/or toxic side effects to a potential therapy, such as immunotherapy. For example, methods of the invention are useful to predict treatment response durability and/or side effects of immune checkpoint inhibitor therapies in patients with cancer, such as melanoma or colorectal cancer.

A first aspect of the disclosure provides an ultrasensitive framework for profiling closely related cell types and states using DNA methylation. Based on methylation data, an analytical platform such as a deep deconvolution algorithm may classify closely related cell states including those within a particular ATM, such as the tumor microenvironment.

A second aspect of the disclosure provides tools for the development of, and the use of, an atlas of cell state-specific methylation profiles. Using purified cells from inhouse and public databases, methods of the invention may be used to identify distinct methylation profiles for a broad range of cellular types (e.g., immune cells, somatic cells, tumor cells, cells originating from various organs) and states (e.g., healthy, diseased, conducive to tumor progression/regression, stressed, pre- or post-treatment). Such an atlas may be used as a reference for deconvolving cell states from any given mixture. In doing so, the atlas may provide a reference used to assess and/or detect one more particular tissue microenvironments in a subject using a liquid biopsy sample.

A third objective of the disclosure is to apply the disclosed assay to multiple conditions leading to an ATM. For example, this may include using ATM methylation profile data to diagnose a disease, such as cancer or a particular type of cancer (e.g., colorectal and melanoma). Similarly, the methylation profile of an ATM can be used to provide insights as to the location of an ATM, such as an organ harboring a tumor or infection. Similarly, methods of the invention may use ATM methylation profiles to assess whether a TME has crossed a threshold from benign to malignant. The presently disclosed methods may also be used to assess TME malignancy even in subjects with low measured tumor mutational burdens (TMB). Early detection of surpassing the threshold to malignancy is critical—as pre-malignant tumors are better candidates for surgical excision and treatment.

In certain aspects, the methods of the invention may be applied on pre-treatment to advanced-stage patients that received a particular therapy, to profile their ATM methylation profile response signatures. These signatures may be used in conjunction with ATM methylation profile data from a patient to predict a durable treatment response and/or toxic side effects. In particular, methylation patterns from cfDNA may provide predictions regarding the treatment response/side effects of immunotherapies or immune checkpoint inhibitors in cancer patients. These predictions may lead to drastic changes in standards of care. For example, although they are often considered a secondary treatment option, in certain cancer patients, immune checkpoint inhibitors provide a dramatic, positive response. Identifying such patients means providing them with the best care possible, without exposing them to potentially deleterious treatment modalities such as radiation and chemotherapy.

In certain aspects, the methods of the invention may be applied on pre-treatment to advanced-stage patients that received a particular therapy, and profile their response signatures. In this way methods of the invention may be used to predict a patient's response to, and the toxicity, of a potential therapy (e.g., an immune checkpoint inhibitor [ICI] or an immunotherapy). For example, the methods assay may be applied on pre-treatment to advanced-stage melanoma patients treated with ICI and used to identify signatures of response and validate those in a held-out test set. In this way, methods of the invention are useful to predict a patient's response to, and the toxicity of, immune checkpoint inhibitors (ICI).

Solid tumors can be considered as having two components: malignant cancer cells and other cells of the body intermixed with the malignant cancer cells. The tumor microenvironment (TME) is complex and plays a critical role in causing inflammation, promoting tumor growth, and/or promoting cell death. The TME may include vascular cells, the cancer cells themselves, non-malignant immune cells, somatic cells surrounding the margin of the tumor, and the extracellular milieu from the cells. See Anderson, 2020, The tumor microenvironment, Current Biol 30(16):R921-R925 and Joyce, 2009, Microenvironmental regulation of metastasis, Nat Rev Can 9(4):239-252, both incorporated by reference. Malignant cells can change the TME in such a way that the immune cells in the TME cannot effectively kill the cancer cells. See Gajewski, 2013, Innate and adaptive immune cells in the tumor microenvironment, Nature Immunol 14(10):1014-1022 and Tumeh, 2014, Pd-1 blockade induces responses by inhibiting adaptive immune resistance, Nature 515(7528):568-571, both incorporated by reference.

Other tissue microenvironments include analogous components to, or themselves are part of, a TME. In much the way a TME is shaped by the activity of the cells, particularly the immune cells, within a TME so too are other aberrant tissue microenvironments, e.g., the inflammatory microenvironment (IME), tissue transplant microenvironment (TTME), a pathogenic microenvironment (PME). Accordingly, methods of the invention may be used to assess, profile, and predict the toxicity/success of therapies directed towards the IME, TIME, and/or PME.

Thus, in certain aspects, the methods of the invention can be used to predict whether a patient will respond, either positively or negatively, to a therapy, such as an immunotherapy.

For example, immune checkpoint inhibitors (ICI) are a promising way to treat certain advanced-stage cancer patients. ICIs block inhibitory receptors on TILs, a phenomenon which is transforming the field of cancer care. However, ICI response in patients remains challenging to predict having success rates ranging from 1% to 60%. Moreover, standard imaging technology cannot assess treatment response reliably at early timepoints. Studies have shown that earlier ICI response assessment can be achieved with serial biopsy analysis of the TME, an inspiring result but clinically not practical. Thus, it is critical to develop a method for monitoring the TME and other ATMs noninvasively using “liquid biopsy”.

Malignant cells can change the TME in such a way that the immune cells in the TME cannot effectively kill the cancer cells. See Gajewski, 2013, Innate and adaptive immune cells in the tumor microenvironment, Nature Immunol 14(10):1014-1022 and Tumeh, 2014, Pd-1 blockade induces responses by inhibiting adaptive immune resistance, Nature 515(7528):568-571, both incorporated by reference. An immune checkpoint inhibitor (ICI) can “take the brakes off” those immune cells and turn them into more potent cancer-killers. The ability of an ICI to kill otherwise unresponsive tumors is showing the potential to transform the treatment of advanced tumors.

However, not all patients benefit from this treatment, and it is challenging to know who will benefit and who will not. Serious side effects with ICI treatment may occur, emphasizing the importance of improving patient selection so that only patients who will benefit get treated. ICI treatment response can be predicted early by tumor biopsy analysis. However, there previously has been no noninvasive method for such a prediction.

The invention provides a computational noninvasive liquid biopsy approach to profile the cellular composition of an aberrant tissue microenvironment. Specifically, methods of the invention are useful to estimate closely related cell types and states including cell states related to tumor profiles from blood plasma derived cell-free DNA and apply it in different clinical settings. Methods of the invention are useful to predict treatment responses and side effects of ICI for patients with cancer such as melanoma or colorectal cancer.

As ICI transform the immune cell compartment of the TME into cancer-killing cells, the treatment response largely depends on the cellular composition of the tumor. See e.g., Thommen 2018, T cell dysfunction in cancer, Cancer Cell 33(4):547-562, incorporated by reference. For example, the TME of a tumor may lack immune cells with cancer-killing potential. Therefore, monitoring the TME before and during treatment would be valuable. However, monitoring the TME has previously required invasive biopsy. Serial biopsy of a patient is not practical and can suffer from sampling bias due to the heterogeneity of the tumor. The invention provides a noninvasive computational end-to-end framework for profiling the tumor microenvironment by liquid biopsy (herein LiquidTMIE) and sequencing of cell-free DNA.

Biopsy is an invasive method where cells are extracted directly from tissue, such as from a tumor, for in-depth examination, and is one of the first steps used by clinicians to diagnose many conditions, such as cancer. Based on analysis of the extracted cells, doctors determine the pathway of treatment. Though this invasive method is the standard practice for solid tumor malignancies, it can be expensive, risky and sometimes impractical. Particularly, monitoring a treatment response precisely would require serial biopsies which is not feasible. Liquid Biopsy is an alternative idea where researchers try to examine tumors from body fluids like blood.

In blood plasma, scientists discovered the presence of cell-free DNA (cfDNA) more than 100 years ago. See Mandel, 1948, Nucleic acids in blood plasma in 1 man, CR Seances Soc Biol Fil 142:241-243, incorporated by reference. As the name cfDNA suggests, these DNA fragments are not contained within cells but rather circulate within blood plasma. When cells die, some of their DNA fragments are released into blood circulation where they can be captured and measured within cfDNA. It is understood that a fraction of those circulating DNA fragments arise from malignant cells in patients with cancer, or similarly stressed cells in other aberrant tissue microenvironments, e.g., the IME, TTME, and PME. These, microenvironment-specific cell-free DNA fragments, when originating from the TME are known as circulating tumor DNA (ctDNA). For clarity, the equivalent circulating DNA originating from diseased or stressed cells in the IME, TTME, and PME are referred to herein as ctDNA.

It is understood that like cancer cells, the TME itself also releases DNA into blood circulation. In cancer, microenvironment-derived cell-free DNA fragments may be referred to as circulating tumor infiltrating lymphocyte TIL DNA (ctilDNA). The IME, TTME, and PME produce equivalents, which will be referred to herein as ctilDNA for clarity.

Though several methods are available to detect ctDNA, this disclosure provides methods capable of detecting or quantifying ctilDNA, including providing its tissue of origin. An objective of this disclosure is to provide a robust computational framework to detect ctilDNA called “LiquidTME” for the liquid biopsy of a particular ATM. One feature significant in cfDNA from TILs is the methylation of CpG dinucleotides.

In DNA, cytosine (C) followed by guanine (G) are known as CpG (the ‘p’ represents the phosphate bond between them). Through an epigenetic mechanism, a methyl (CH₃) group is added to the C of a CpG site. This phenomenon is known as CpG methylation. It turns out that different cell types and states have specific methylation patterns that regulate gene expression. To quantify methylation patterns in DNA, bisulfite treatment followed by next-generation sequencing is commonly used. Briefly, in this bisulfite-based sequencing method, if a C in a CpG site is not methylated, the C converts to Uracil (U) which is subsequently recognized as Thymine (T). On the other hand, if a C in CpG site is methylated, the C remains as it is. Finally, the sequenced reads are aligned to the reference genome and for every CpG position, the ratio of C and T is calculated. Recent studies have shown potential utility of cfDNA methylation to provide early detection of certain diseases and conditions, such as cancer, including determining the tissue of origin. However, TILs have not previously been profiled using CpG methylation from cfDNA. The disclosure provides methods useful to profile ctilDNA using CpG methylation of cell-free DNA by developing a novel liquid biopsy approach. These profiles may be used to assess the composition of an ATM and provide a predictive assessment of a certain disease or condition.

Literature on ctDNA detection suggests certain considerations, which find equal applicability to analogous cfDNA emanating from non-tumor ATMs.

Circulating tumor DNA or ctDNA are DNA fragments coming from cancer cells. Cancer is a disease that begins with genomic mutations. Based on tracking these mutational signatures, ctDNA can be quantified from cfDNA sequencing. While several ctDNA detection technologies exist, they generally work by querying genomic positions likely to be mutated in cancer cells and deeply sequencing these positions (known as targeted sequencing) in plasma cfDNA. After targeted sequencing, the pre-defined genomic positions are interrogated for mutations, and in this way ctDNA molecules are detected and quantified. See Newman, 2014, An ultrasensitive method for quantitating circulating tumor DNA with broad patient coverage, Nature Med 20(5):548-554, Forshew, 2012, Noninvasive identification and monitoring of cancer mutations by targeted deep sequencing of plasma DNA, Science Trans Med 4(136):136ra68-136ra68, and Murtaza, 2013, Non-invasive analysis of acquired resistance to cancer therapy by sequencing of plasma DNA, Nature 497(7447):108-112, all incorporated by reference.

It is also understood that ctDNA detection is also affected by background noise. Noise can be introduced during sample preparation and sequencing. This can confound results when quantifying rare ctDNA fragments in patients with low burdens of disease (e.g., early-stage cancer or post-curative-intent-treatment minimal residual cancer). See Chaudhuri, 2017, Early detection of molecular residual disease in localized lung cancer by circulating tumor DNA profiling, Cancer discovery, 7(12):1394-1403, Chin, 2019, Detection of solid tumor molecular residual disease (MRD) using circulating tumor DNA (ctDNA), Molecular Diag Ther 23(3):311-331, and Newman, 2016, Integrated digital error suppression for improved detection of circulating tumor DNA, Nature Biotech 34(5):547-555, all incorporated by reference. Duplex variant support, where both positive and negative strands are sequenced, and the mutated variant is corroborated in both parent strands of DNA, reduces this noise significantly. However, requiring duplex variant support is an inefficient approach as 80-90% of recovered cell-free DNA sequencing reads are typically single-stranded without duplex support. Another approach to reduce noise is to profile the background error pattern by sequencing healthy donor-derived cell-free DNA and to account for it while querying mutations in patient cfDNA. Other work has shown it is possible to reduce background noise by requiring co-detection of adjacent mutations within the same cfDNA fragment.

The invention uses methylation-based cell-free DNA analysis to detect circulating DNA from a particular ATM and predict its tissue of origin. In particular, methods of the invention may use methylation-based cell-free DNA analysis to detect cfDNA from an ATM and predicts its tissue of origin. For example, methods of the invention may use ctDNA to predict the tissue of origin of a tumor. For background, see Xu, 2017, Circulating tumor DNA methylation markers for diagnosis and prognosis of hepatocellular carcinoma, Nature materials, 16(11):1155-1161, Moss, 2018, Comprehensive human cell-type methylation atlas reveals origins of circulating cell-free DNA in health and disease, Nature Comm 9(1):1-12. Shen, 2018, Sensitive tumor detection and classification using plasma cell-free DNA methylomes, Nature 563(7732):579-583, Guo, 2017, identification of methylation haplotype blocks aids in deconvolution of heterogeneous tissue samples and tumor tissue-of-origin mapping from plasma DNA, Nature Genetics 49(4):635-642, and Li, 2018, CancerDetector: ultrasensitive and non-invasive cancer detection at the resolution of individual reads using cell-free DNA methylation sequencing data, Nucleic Acids Res 46(15):e89-e89, all incorporated by reference. Methylation-based ctDNA detection mainly has two steps: (1) like mutational signatures, cancer-specific methylation signatures are first identified; (2) deconvolition techniques are used to detect the ctDNA are used to detect the ctDNA and infer tumor tissue of origin. By extension, as the presence of base mutations are not required, methylation-based detection of cfDNA originating from non-cancerous ATMs is likewise possible.

Using differentially methylated CpG sites, the tissue origin of ctDNA can be identified. Instead of single CpG sites, co-associated adjacent CpG sites (which have been dubbed Methylation Haplotype Blocks or MHBs), can be used to more accurately deconvolve methylation data. Differentially methylated MHBs are identified, and ctDNA and tissue of origin from cfDNA may be identified using a tool such as a random forest classifier. Another approach involves classifying aligned reads individually with the help of methylation patterns of each read. CancerDetector [Li 2018] may be useful in such a method whereby using on a beta binomial model, every cfDNA sequencing read is assigned as either cancer-derived or not cancer-derived.

As discussed above, tumor-infiltrating leukocytes (TILs) (and equivalents in other microenvironments) are leukocytes (white blood cells) that infiltrate the tumor and contribute to the composition of the tumor microenvironment. Based on the immune cells in the TME, tumors can fall broadly into three major classes: immune-infiltrated, immune-excluded and immune-silent. In the immune infiltrated case, TILs infiltrate the tumor and become resident within the tumor tissue. When TILs are found only on the border of the tumor, that tumor is classified as immune-excluded. Finally, some tumors are completely devoid of immune cells and are categorized as immune-silent.

Considering one objective of the disclosure is to detect TIL content from cfDNA, the molecular profile of TILs must be different from PBLs. Using ATAC-seq, it is possible to obtain distinct epigenetic programs in microenvironment-specific immune cells, such as tumor-specific CD8 T cells. See Philip, 2017, Chromatin states define tumor-specific t cell dysfunction and reprogramming, Nature, 545(7655):452-456, incorporated by reference.

The present invention uses the insight that the methylation profiles of certain cells, particularly immune cells, coming from a particular aberrant microenvironment differ from those of normal cells, unassociated with the microenvironment. Thus, methods of the invention can be used to detect the presence of and/or assess an aberrant tissue microenvironment, e.g., a TME, based on detecting the presence of nucleic acids with a methylation profile correlated with cells, such as immune cells, emanating from such a microenvironment.

For example, it has been shown that the methylation profile of CD8 T cells coming from tumor tissue is different than that of normal CD8 T cells. See Yang, 2020, Distinct epigenetic features of tumor-reactive cd8+t cells in colorectal cancer patients revealed by genome-wide DNA methylation analysis, Genome Biol 21(1):1-13, incorporated by reference. The gene promoter from CD8 T cells isolated from the TME of are hypomethylated for the tumor-reactive marker genes CD39 and CD103. Using such epigenetic patterns. From those insights, methods of the invention are useful to distinguish TILs from PBLs using methylation. By extension, methods of the invention are able to determine whether cfDNA originates from an ATM or normal tissue.

Heterocellular tissue consists of different cell types and states. In certain aspects, deconvolution methods are used to computationally estimate the cellular proportions of these different cell types from bulk sequencing data. Tissue deconvolution was developed primarily for gene expression data where gene expression of the tissue is modeled as a weighted sum of the gene expression of underlying cell types. CIBERSORT is a popular such method that first identified signatures from 22 cell types and then used support vector regression to estimate those 22 cell types from bulk expression data. See Newman, 2015, Robust enumeration of cell subsets from tissue expression profiles, Nature Meth 12(5):453-457, incorporated by reference.

CIBERSORTx is a recent extension of CIBERSORT that provides the ability to build signature matrices from single-cell RNA-sequencing data and to profile distinct cellular states (e.g., exhausted vs. non-exhausted CD8 T cells) within each deconvolved cell type. See Newman, 2019, Determining cell type abundance and expression from bulk tissues with digital cytometry, Nature biotechnology, 37(7):773-782, incorporated by reference.

The idea of deconvolution can be extended from gene expression data to ATM methylation profile data to ascertain the status of the underlying cell types, and thus provide an assessment of the ATM. In certain aspects, such deconvolution includes considering methylation status of CpG sites as a weighted sum of the methylation status of the underlying cell types. Based on those insights, MethylCIBERSORT [Chakravarthy, 2018, Pan-cancer deconvolution of tumor composition using DNA methylation, Nature Comm 9(1):1-13, incorporated by reference] uses CIBERSORT applied to methylation sequencing data whereas MethylResolver [Arneson, 2020, MethylResolver—a method for deconvoluting bulk DNA methylation profiles into known and unknown cell contents, Communications biology, 3(1):1-13, incorporated by reference] uses Least Trimmed Squares regression for methylation deconvolution. The invention uses such suitable methods for the deconvolution of methyl-seq data. In addition to modelling the deconvolution e.g., as a system of linear equations, the invention may include the use of machine learning classifiers that are trained on some bulk samples and then applied to held-out data.

The disclosure provides a new platform for noninvasive profiling of TILs from patients. In preferred aspects, noninvasive profiling of TILs includes decoding TIL-derived methylation signatures identified from plasma-derived cell-free DNA molecules. Such signatures may, for example, indicate a disease status, e.g., remission, recurrence, minimal residual disease. Signatures may also be predicative of a certain patient's expected response durability and/or toxicity to a potential therapy. Similarly, the signature may be indicative of a favorable ATM for disease progression. In the context of cancer, this may include assessing methylation signatures from TILs identified as emanating from a tumor or tumor margin and/or from non-TME cells. Using detected profiles of cells from the margin and/or non-TME cells may provide loci indicative of tumor progression or regression (e.g., via somatic regression). Such information provides critical insight when forming treatment plans.

In certain aspects, systems of the invention are used to deconvolve methylation data and construct, contribute to, or use a methylation cell atlas that may include reference data for several different human cell types and states using both in-house and public data resources (like BLUEPRINT, ENCODE). That platform is useful for immunotherapy response and toxicity assessment. Embodiments make use of banked and de-identified biospecimens of individuals with a known condition, e.g., such as those available from the Yale SPORE in Skin cancer (YSPORE-SC). Those specimens, which include, for example, melanoma biopsies, plasma samples, and peripheral blood leukocyte samples, have been collected with the informed signed consent of participants according to Health Insurance Portability and Accountability Act (HIPAA) regulations.

As methods of the invention contribute to such atlases, the atlases may grow to encompass broad cell types and/or cell states with methylation profiles indicative of a variety of ATMs correlated with certain diseases or conditions. For example, the atlas may include methylation patterns associated with inflammatory conditions (e.g., macular degeneration and rheumatoid arthritis), autoimmune diseases, transplanted tissue (including rejection and graft versus host conditions), pathogenic diseases, immune responses, and other conditions. Consequently, with a sufficient atlas, methods of the invention may be used as a pan-diagnostic of a few or many conditions.

The algorithm to deconvolve methylation data with high sensitivity and specificity may use any read assembly, alignment, or mapping tools. Many alignment tools of methylation data produce BedGraph files as the final output and most traditional deconvolution tools work on the BedGraph file format. See Hoang, 2014, CWig: compressed representation of Wiggle/BedGraph format, Bioinformatics 30(18):2543-2550 and Kent, 2010, BigWig and BigBed: enabling browsing of large distributed datasets, Bioinformatics 26:2204-2207, both incorporated by reference. However, some embodiments of the invention operate with a sequence alignment map (SAM) or binary alignment map (BAM) file format [Li, 2009, The sequence alignment/map (SAM) format and SAMtools, Bioinformatics 25:2078-2079, incorporated by reference, incorporated by reference] as doing so may provide deeper read-level sequencing data. In a BAM file, aligned reads of a mixture are stored. And as a read-pair (i.e., DNA fragment) of a mixture generates from a constituent single cell, classification based on fragment will provide more power to deconvolve the mixtures. Therefore, the aligned DNA fragment of a given mixture will be classified preferably using a SAM or BAM file from the fragment.

In a BedGraph file, for every CpG site the average methylation values are calculated after aligning the read to the corresponding reference genome. While this is a reasonable approach, it may miss an aspect which is the pattern of methylation within a read. On the other hand, BAM files contain aligned reads with read-level information, which allows for the capture of read-level patterns and to use those to deconvolve the mixture on a per-fragment level.

Based on the above observation, the invention provides a method to deconvolve methylation bulk mixtures into purified cell states.

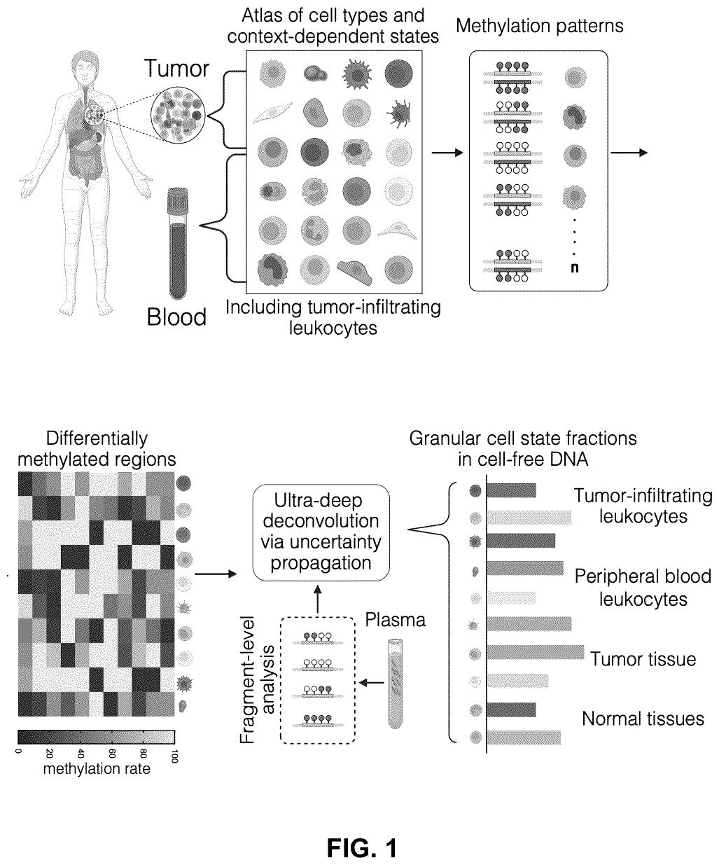

FIG. 1 shows steps of an exemplary method to deconvolve bulk methylation data into data representative of purified cell states. First, differentially methylated regions (DMRs) are identified for cell types and/or cell states of interest. These per-cell-state DMRs serve as specific signatures. To generate these cell state specific signatures, a cell state is compared against all other cell states in an atlas or database, traditionally known as one vs. rest comparison. However, to deconvolve a multitude of cell states (≥20), several of which are closely related to one another, provided are methods to generate a better signature for each cell state.

Methylation profiling of purified cell types and context-dependent cell states are first obtained to identify differentially methylated regions and generate the signature matrix. Those regions are enquired in bulk methylation mixtures to deconvolve closely related context-dependent cell types and states. Given a signature matrix of cell states of interest, every read pair or fragment of a mixture is tested one by one. Given that each sequencing read is obtained from a fragment that comes from a single cell, it is preferable to use a deconvolution method that quantifies on a per-fragment level.

While testing a fragment, the fragment methylation pattern is tested for match with the predefined signature matrix. If it matches with a specific cell state's signature, that fragment is classified to that cell state. Finally, to get the cellular fraction, all the fragments classified to that cell state are counted and divided by the number of available fragments tested for that cell state. That method is herein dubbed Read Counting. As proof of principle, 6 purified cell types were taken from the BLUERPINT public database and prepared 25 in silico mixtures. Read Counting shows a good correlation with ground truth for those mixtures. See Fernández, 2016, The BLUEPRINT Data Analysis Portal, Cell Syst 3(5):491-495, incorporated by reference.

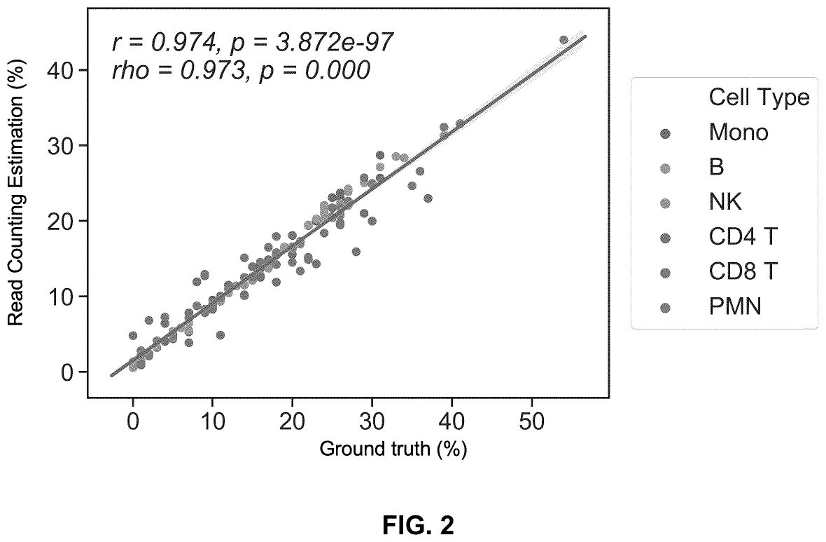

FIG. 2 gives results showing the performance of Read-Counting. As proof of principle using 6 cell types, 25 in silico mixtures are prepared where Read Counting shows strong correlation with known proportion. One critical parameter of the Read Counting approach is how many CpG sites are desired to consider for each fragment. This is important because increasing the number of CpG sites required per fragment decreases the false positive rate.

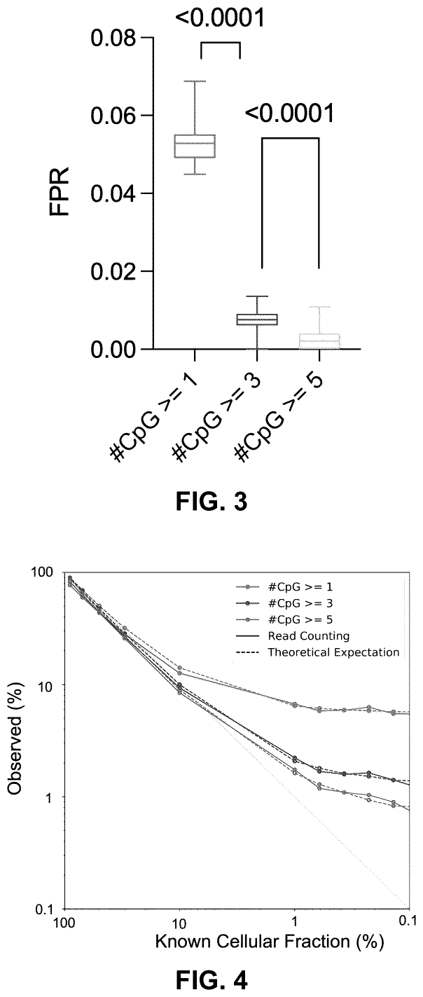

FIG. 3 shows decreasing false positives with increasing number of CpG sites. However, if the number of CpG sites required per fragment is too high, the number of available fragments may decrease such that sensitivity of the resulting deep deconvolution approach is too low.

FIG. 4 compares read counting to different numbers of CpG sites. Though multiple CpG sites per fragment help to achieve a lower limit of detection (bottom line in FIG. 4), the number of available fragments with multiple CpG sites decreases.

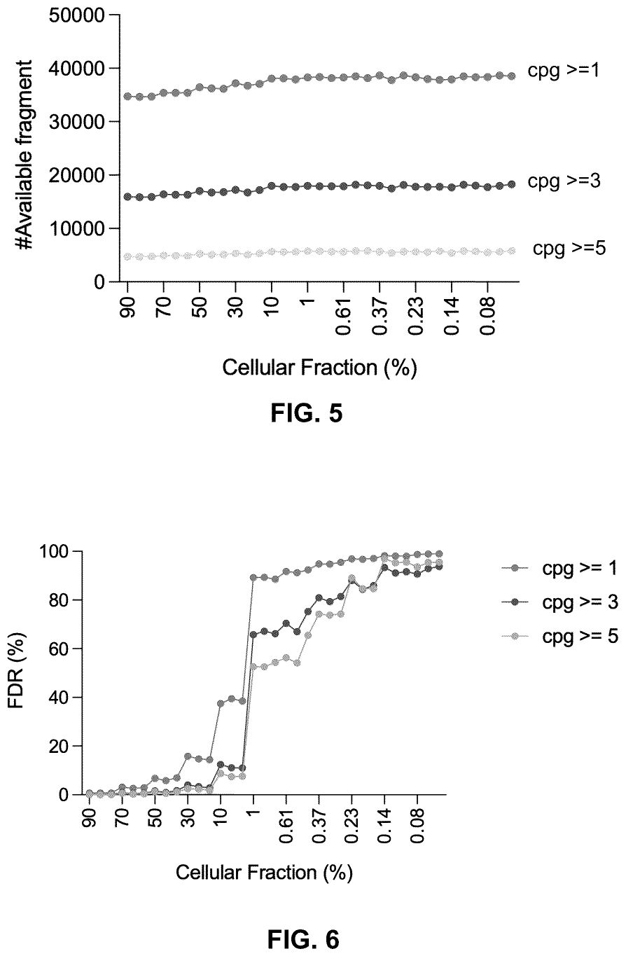

FIG. 5 shows that the number of available fragments is lower with multiple CpG sites per fragment.

FIG. 5 shows that, on the other hand, if all available fragments (CpG≥1) are considered, there is more than 80% False Discovery Rate (FIG. 6) below 1% of cellular fraction. Considering all these observations, the method may be optimized by implementing the following measures: instead of taking any fixed minimum CpG cutoff, consider every fragment but give more weightage to a fragment that has more CpG sites. That approach will consider all fragments along with the benefit of multiple CpG sites. Several different cell types and cell states can be deconvolved. Those cell types and states may be very closely related, and their signatures may be difficult to distinguish. In that case, it may be useful to consider the discrimination power of a CpG for a given fragment. The discrimination power of a CpG site lies in the fact that each such site creates and defines a distance between the cell type/state of interest and others.

The limit of detection analysis shows that below 1% there is substantial noise. From the predefined signature matrix, it is possible to generate the true positive rate (TPR) and the false positive rate (FPR). As the theoretical expectation shows that one can predict the Read Counting result using binomial modelling, it is expected to be possible to reduce the noise by accommodating the signature matrix's FPR in the Read Counting algorithm by using a Monte Carlo based approach.

FIG. 3 through FIG. 6 show a technical assessment of Read Counting.

FIG. 3 is a Box plot showing that a higher number of CpG sites per fragment results in a lower false positive rate (FPR).

FIG. 4 shows the results of a Limit of detection analysis for a cell type (Neutrophil) within an in silico mixture. With higher numbers of CpG sites, better detection limit is achieved as FPR decreases. Theoretical expectation is shown as a dashed line. FIG. 5 graphs the decreasing number of available fragments as the CpG cutoff is raised. FIG. 6 shows change of FDR with cellular fraction for different numbers of CpG sites per fragment.

Methods of the invention may use, and the results may contribute to, a database or atlas of cell state-specific methylation profiles correlated with particular types of ATM and ATM states. Methods may use existing (or copies of existing) such resources including, for example, those dubbed scMethBank, m5C-Atlas, the DNAm-atlas, and MethAtlas. See for example Zong, 2022, scMethBank: a database for single-cell whole genome DNA methylation maps, Nucleic Acids Research 50(D1):D380-D386, Ma, 2022, m5C-Atlas: a comprehensive database for decoding and annotating the 5-methylcytosine (m5C) epitranscriptome, Nucleic Acids Res 50(D1):D196-D203, Zhu, 2022, A pan-tissue DNA methylation atlas enables in silico decomposition of human tissue methylomes at cell-type resolution, Nat Meth 19:296-306, Loyfer, 2022, A human DNA methylation atlas reveals principles of cell type-specific methylation and identifies thousands of cell type-specific regulatory elements, bioRxiv:477547 (28 pages), Moss, 2018, Comprehensive human cell-type methylation atlas reveals origins of circulating cell-free DNA in health and disease. Nat Commun 9:5068, and Katsman, 2022, Detecting cell-of-origin and cancer-specific methylation features of cell-free DNA from Nanopore sequencing, Genome Biol 23:a158, all incorporated by reference.

In the LiquidTME framework as shown in FIG. 1, differentially methylated regions or the signature matrix is another important component that the deconvolution algorithm uses as a reference to match. Traditionally, the signature for a cell type is generated by a one vs. rest fashion wherein, while preparing a cell type's reference, all other cell types are considered together.

More specifically, the cell types are split into two groups. One group has the cell type of interest and the other has the rest. Though this technique is efficient and reasonable for smaller numbers of cell types that are clearly distinct from one another, if the signature matrix has lots of closely related cell types and states (e.g., from public and/or in-house data), the one vs. rest fashion can become problematic. For the atlas, it may be useful to develop a methylation signature matrix that will include several different cell types and states, many of which will be closely related. In this scenario, instead of using the one vs. rest approach, methods use a tiered approach where the existing biological information will be used. Specifically, certain steps are used to generate the methylation signature matrix.

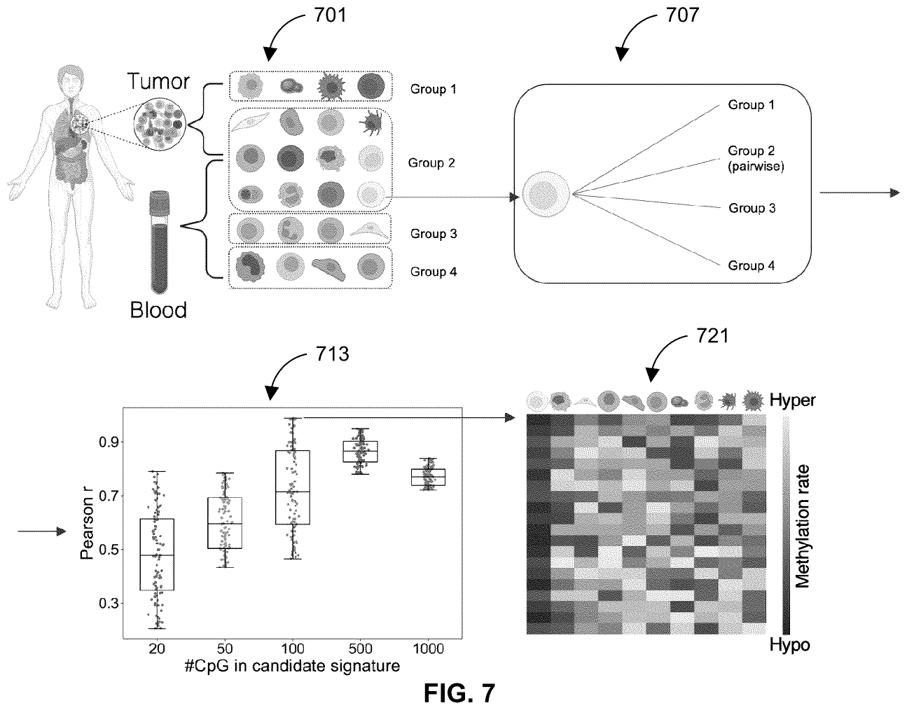

FIG. 7 gives a workflow of signature matrix generation. At step 701, the workflow starts by considering the methylation profile of different cell types (e.g., immune cell and somatic cells) and states (e.g., immune cell exhaustion or expansion) in the human body.

Cell types are first grouped based on known biology. At step 701, all cell types and states will be grouped into smaller groups based on biological similarity so that closely related cell types/states are grouped together. For example, CD4 T, CD8 T and Treg cell states are all T cells, thus those are grouped together in a single group. This group information will be user-defined so that based on the context, the granularity may be adjusted (step 701 in FIG. 7). In this groupwise framework, all cell states belong to a unique group. When the profile of a cell state is generated, the groups are of two kinds. The group that includes that cell state is dubbed the Own Group and other groups as Rest Groups. To generate profile of a cell state, the methylation distance between that cell state and all Rest Groups will be calculated separately.

At step 707, for any cell type/state such as the last one depicted in Group 2 in step 701, the distance from each group is measured. For Group 2, distance for all cell states are measured separately as that last cell is biologically similar to rest of the cells of Group 2. In step 713, with different distances and CpG number thresholds, different candidate signatures are obtained for the cell. Step 721 gives the optimal signature for the cell state from the previous step where columns are cell types/states and rows are CpG positions. The first column cell is mostly light (from the hyper- hypo-scale) as hypomethylation is being used as the cell state-specific signature. It is possible to compare with all cell states of that group one by one as they are closely related (step 707 in FIG. 7).

Third, after all the groupwise distance comparisons, candidate signatures may be combined using different distance thresholds. These combinations are the candidate signatures for a cell state. To find out the optimal signature, in-silico mixtures are generated for training. As testing all the candidate signatures can be time consuming, an option is to take a subset of them based on how many CpG sites are available in a candidate signature (step 713). Finally, the signature that maximizes the performance within in-silico training mixtures is identified (step 721).

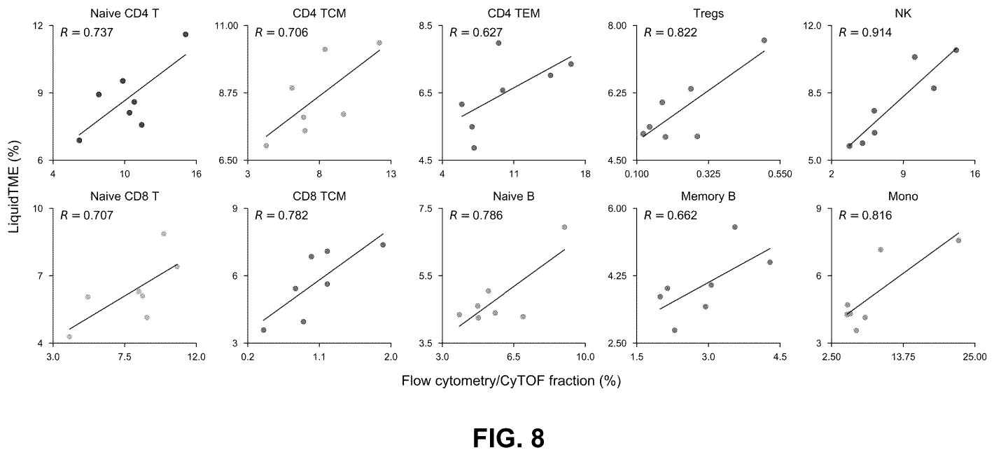

FIG. 8 shows results from LiquidTME-based deep deconvolution. Comparison between LiquidTME and wet lab ground truth for 10 immune cell subsets from the Methylation Cell Atlas in peripheral blood from 7 healthy human subjects is made. In this non-limiting example, for wet lab ground truth, flow cytometry or time-of-flight cytometry (CyTOF) is used.

The above approach may be extended for use in all cell types and states in the atlas (e.g., ≥50) to generate a large signature matrix which may be referred to as the Methylation Cell Atlas. The disclosed method of read counting combined with use of this Methylation Cell Atlas is dubbed LiquidTME, indicating the ability to profile ATM and the immune cells therein from a liquid biopsy sample.

Using a database of cell states and their epigenetic markers such as the methylation cell atlas, methods of the invention may be used to provide several types of predictive analysis using methylation data from a liquid biopsy sample. Methods of the invention preferably include identifying epigenetic modifications in nucleic acid from a sample such as by bisulfite sequencing or enzymatic methyl sequencing cfDNA from a blood or plasma sample. Patterns in the epigenetic modifications are compared to the database or atlas or read by a machine learning algorithm.

Using a database of cell states and their epigenetic markers such as the methylation cell atlas, one may perform a method of predicting an effect of therapy. Methods of the invention are useful to predict immunotherapy toxicity (and treatment toxicity more generally) from cell-free DNA methylation cell state analysis. For example, from a liquid biopsy sample, methods of the invention may use cfDNA to granularly profile the immune cell repitoire, which may include immune cell diversity, activation, and/or abundance. That measured abundance and/or diversity forms the basis of a prediction of immunotherapy toxicity or response.

In certain preferred embodiments, methods include measuring the abundance of activated CD4 T effector memory (TEM) cells to assess immunotherapy-related adverse events (irAE) in patients with cancer. Toxicity prediction is based on the fact that abundant activated CD4 TEM cell levels are associated with severe irAE development. Certain embodiments may also measure TCR diversity from the patterns in the epigenetic modifications. Higher TCR clonotype diversity in bulk peripheral blood is understood to be predictive of severe irAE development. When one or both of TEM abundance and TCR diversity exceeds a threshold in a pre-ICI patient, a prediction of irAE is made. Preferred embodiments use a composite model integrating both features-activated CD4 TM cell abundance and bulk TCR diversity. In some embodiments, the composite model yielded an AUC of 1.0 in bulk cohort 2 (P=0.04) and an AUC of 0.86 by leave-one-out cross-validation (LOOCV) for predicting severe irAE.

For ground-truthing and to build the cell state atlas, methods may include droplet-based scRNA-seq and time-of-flight mass cytometry of peripheral blood from patients treated with a particular therapy. In an exemplary embodiment, methods include droplet-based scRNA-seq and time-of-flight mass cytometry of peripheral blood from cancer patients treated with combination immunotherapy (anti-PD1/anti-CTLA4) to determine the activated CD4 memory T cell subset most predictive of severe irAE development.

Time-of-flight cytometry and bulk RNA sequencing (deconvolved with CIBERSORTx and TCR-assembled using MiXCR) may be performed to validate the composite model (activated CD4 memory T cell abundance integrated with TCR diversity). CIBERSORTx is a machine learning tool that infers cell-type-specific gene expression profiles. See Newman, 2019, Determining cell type abundance and expression from bulk tissues with digital cytometry, Nat Biotech 37:773-782, incorporated by reference. MiXCR is a software package for immune profiling. See Bolotin, 2015, MiXCR: software for comprehensive adaptive immunity profiling, Nat Methods 12(5):380-1, incorporated by reference. RNA sequencing and or mass spectrometry with gene expression and immunity profiling may be used to provide ground truth or gold standard data.

Using such gold standard data may show that gene expression signatures of irAE toxicity and immunotherapy response can be inferred from promoter-level methylation of cell-free DNA to enable prediction of both irAE severity and immunotherapy response from pre-treatment plasma. That provides liquid biopsy prediction of both immunotherapy response and toxicity from a single cell-free DNA assay. To validate such predictions, cell-free DNA is extracted from pre-treatment plasma (cycle 1 day 1) patients in tested in the ground-truthing (e.g., with scRNA-Seq, TOF mass cytometry, and bulk RNA seq) subject to methylation sequencing, and queried over the gene sets previously reported (Lozano, 2022, Nat Med 28:353, incorporated by reference) to be associated with immunotherapy toxicity (CD4 T 5+35) and response (CE9 ecotype32). Pre-treatment promoter methylation signatures are correlated with irAE severity and immunotherapy response.

### Examples

### Validation of LiquidTME

The following examples show the performance of methods of the disclosure used to accurately predict TIL content within an ATM, in this case tumor masses, noninvasively via plasma cfDNA analysis. To test the performance of LiquidTME, a less granular version of LiquidTME is implement where instead of comparing all cell states of own group separately, states are combined and group-wise comparison are conducted. Methylation data for 22 purified cell states were collected from the BLUEPRINT public database [See Fernández, 2016, The BLUEPRINT Data Analysis Portal, Cell Syst 3(5):491-495, incorporated by reference] and used to generate a signature matrix of these 22 cell states. Using the generated Signature Matrix or Methylation Cell Atlas, seven real BULK PBMC samples were used for wet lab ground truth for some cell states using Flow cytometry or CyTOF (FIG. 8). In addition to using wet lab ground truth for multiple cell states, an in-silico mixture is useful to assess the performance.

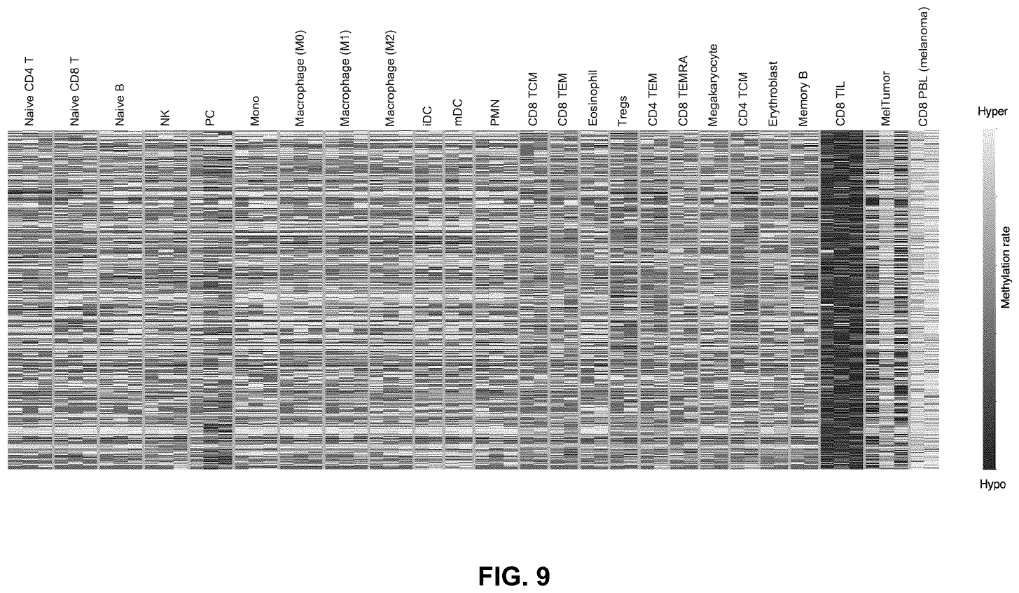

FIG. 9 shows a signature for CD8 TIL. Columns are different cell types/states and rows are CpG positions. CD8 TILs display a distinct pattern compared to other cell types and states.

Moreover, using this tiered signature matrix generation approach, signatures for TILs, specifically CD8 TILs have been generated. The purified CD8 TILs, tumor cells from melanoma patients (MelTumor) and CD8 PBL (normal CD8 PBLs from melanoma patients) are isolated and methylation-sequenced. The examples show the performance of methods herein to accurately predict TIL content within tumor masses noninvasively via plasma cfDNA analysis.

In addition to BLUEPRINT and in-house data, more cell types and states from other public resources are included and used in preparing a comprehensive methylation cell atlas. As discussed above, if the signature for closely related cell states is not distinct and deconvolution approach faces trouble, problematic cell states are compared pairwise. Besides generating profiles from methylation data, the methylation profile may be predicted from scRNA-seq data if necessary. There are studies which show that there is a negative correlation between promoter methylation and gene expression levels. See Anastasiadi, 2018, Consistent inverse correlation between DNA methylation of the first intron and gene expression across tissues and species, Epigenetics & chromatin, 11(1):1-17, incorporated by reference. As methylation data is being collected from purified cells, predicting methylation profiles from scRNA-seq may be useful.

### Non-Invasive TIL Profiling of Multiple Cancer Types

The presently-disclosed methods are able to accurately profile TILs and thus profile the ATM (tumor) from which they originated. This may include, for example, determining the type of cancer creating a TME detectable via epigenetic modifications found in cfDNA. Further, the TIL methylation profile can be used to provide a prediction of response durability and/or toxicity to a therapy, in this case, an immunotherapy.

To assess whether noninvasively profiling tumor infiltrating leukocytes (TIL) will have utility in vivo, it is important to compare estimated TIL composition in the plasma of colorectal (CRC) and melanoma patients against orthogonal measures of TIL content in paired tumors (e.g., by flow cytometry). Banked viably preserved tumor, plasma, and PBL samples from 30 patients with advanced melanoma are analyzed. Patients with matched epidemiological and clinical characteristics are analyzed with no deliberate attempts to exclude certain genders/sexes or minority groups. A subset of patients has undergone tumor biopsy and blood draw pre-treatment. A subset of patients with relapse specimens is also assessed, enabling evaluation of changes in TIL content from baseline. In parallel, banked whole blood samples (plasma and PBLs) from 10 age-matched healthy non-pregnant donors (who should have no TILs present) are obtained from a local blood bank without regard to demographic features or certain genders/sexes will be processed. In addition to noninvasive TIL profiling, methods of the invention are applied to predict toxicity in melanoma patients.

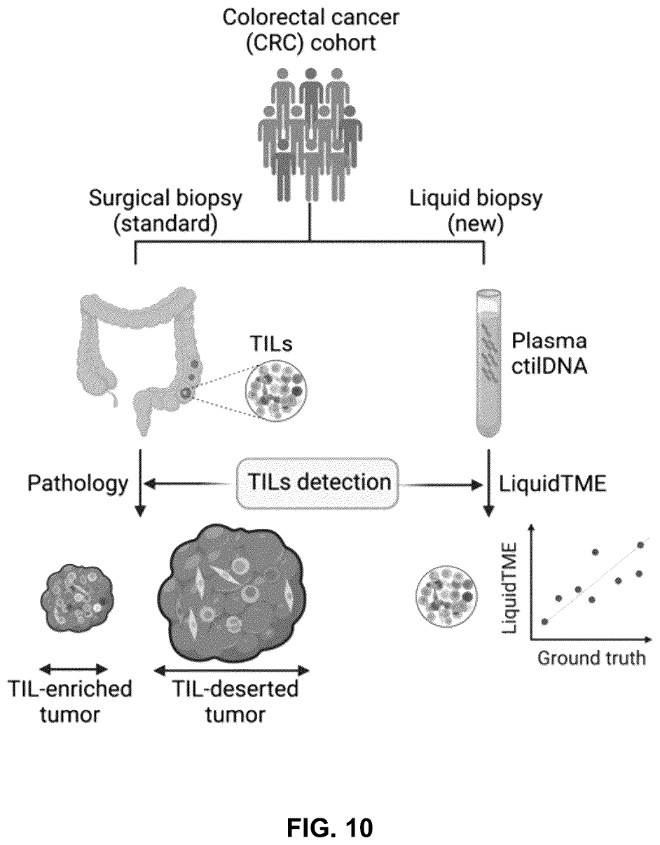

FIG. 10 shows application to CRC cfDNA and specifically how LiquidTME applied to blood plasma derived cfDNA is compared to ground-truth TIL content (measured by analysis of the tumor biopsy).

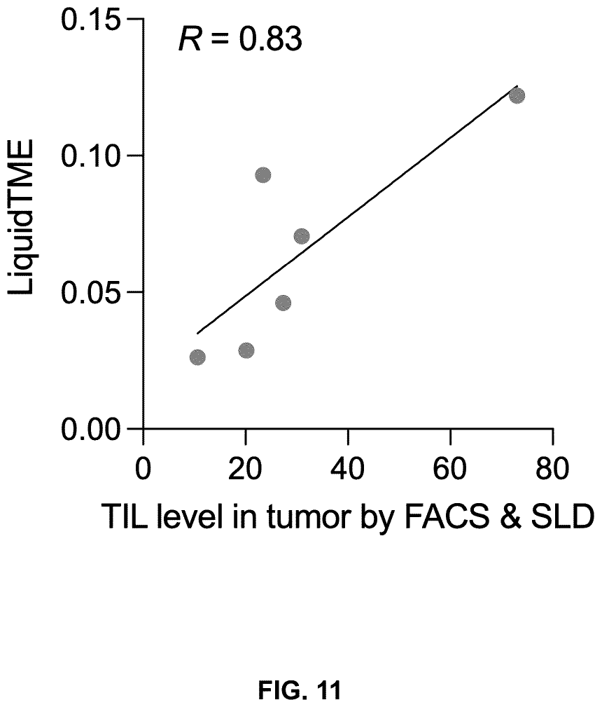

FIG. 11 shows a correlation of LiquidTME-estimated TIL content from cfDNA with the paired tumor biopsy result. The TIL level in tumor is obtained by standard FACS and SLD imaging techniques.

As proof of principle, the TIL signature is taken by methods of the disclosure and tested on 6 CRC patients' cfDNA where matched bulk tumor is available. It was hypothesized that the TIL content in bulk tumor tissue should correlate with ctilDNA levels quantified by the LiquidTME approach. Indeed, the method shows a significant positive correlation with wet lab ground truth (FIG. 11).

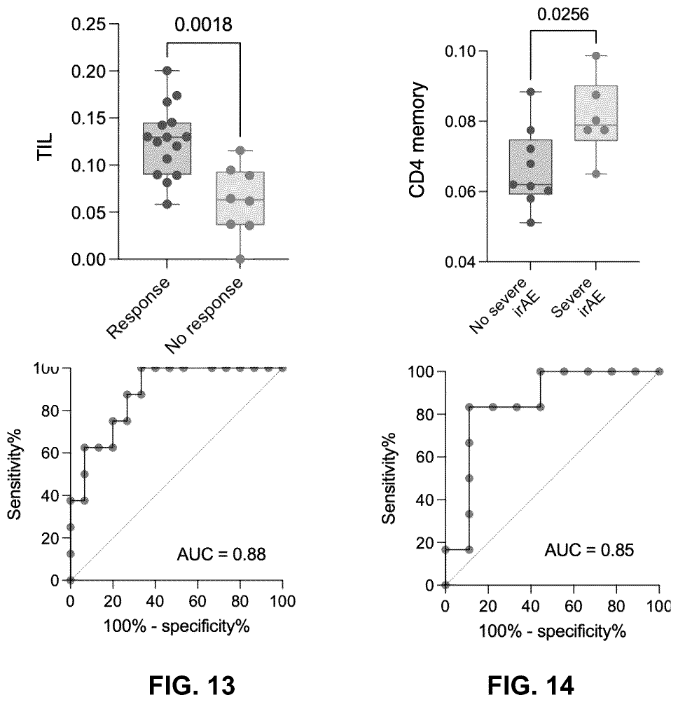

Next, methods of the disclosure are applied to 23 melanoma patients' plasma cfDNA to detect TIL content noninvasively and predict response to immunotherapy. All these patients had a diagnosis of metastatic melanoma and received immunotherapy. Results show that ctilDNA is higher in patients who responded to the treatment (FIG. 13).

This response prediction task is modeled as a machine learning problem to improve performance in a larger cohort. This enables use additional cell-free DNA features (such as fragment length) to improve response prediction. Studies show that ctDNA fragment length distribution is different than healthy cfDNA. Studies have also shown that ctDNA fragments have different end motifs than healthy cfDNA. See Underhill, 2016, Fragment length of circulating tumor dna, PLoS genetics, 12(7):e1006162, Mouliere, 2018, Enhanced detection of circulating tumor DNA by fragment size analysis, Science Trans Med 10(466):eaat4921, Cristiano, 2019, Genome-wide cell-free DNA fragmentation in patients with cancer, Nature, 570(7761):385-389, and Mathios, 2021, Detection and characterization of lung cancer using cell-free DNA fragmentomes, Nature Comm 12(1):1-14, all incorporated by reference. The ctilDNA fragment length distribution is found to be different, as it too is coming from the TME, and is thus different from healthy cfDNA. Fragment length may be integrated as another feature in the predictive model.

### Toxicity Prediction

In this example, methods of predicting immune-related adverse events were tested. Methods of the invention were able to determine TCR repertoires for an ATM, and using that information, predict treatment response durability and toxicity. In this non-limiting example, immune-related adverse events were tested using samples from a cohort of 15 patients. That cohort of 15 patients was previously subject to profiling of bulk TCR beta repertoires in peripheral blood monocyte PBMC samples by immunoSEQ and RNA-Seq as described in Lozano, 2022, Nat Meth 28(2):353-362, incorporated by reference. Those results provide a ground truth for testing results of the LiquidTME methodology.

Immunotherapy can be toxic which is known as immune-related adverse events (irAE). A recent study in Nature Medicine showed that higher activated CD4 memory T cell levels in peripheral blood predicted higher rates of severe irAE in melanoma patients. See Lozano, 2022, T cell characteristics associated with toxicity to immune checkpoint blockade in patients with melanoma, Nature Med 28(2):353-362, incorporated by reference.

The present invention includes an ultra-sensitive deconvolution method to classify closely related cell states and a comprehensive methylation cell atlas. Methods of the disclosure are able to detect activated CD4 memory T cell levels from cfDNA. To test this, toxicity information was collected from the 15 patients in the melanoma cohort of Lozano 2022 and applied the LiquidTME method. Methods herein provide a framework to infer constituent cellular proportions from cell-free DNA methylation data. Developing a novel deconvolution algorithm along with a comprehensive methylation cell atlas, methods of the invention are able to profile the TME noninvasively for the first time. These methods are feasible given these preliminary experiments.

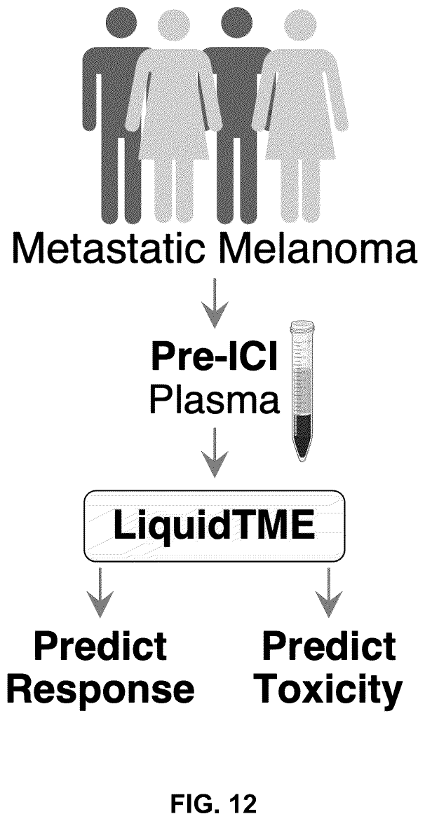

FIG. 12 through FIG. 14 show the application to melanoma cfDNA.

FIG. 12 shows that before the start of ICI in melanoma patients, LiquidTME is applied to the patients cfDNA to predict response and toxicity.

FIG. 13 is a box plot and ROC plot showing prediction of patients' response.

FIG. 14 shows the box plot and ROC plot for toxicity prediction using CD4 memory cell estimated by LiquidTME. The p-values of box plots calculated by 2-sided MWU test.

FIG. 14 shows that LiquidTME estimates higher CD4 memory cell in patients with severe toxicity. Based on this showing, LiquidTME is concordant with other sequencing approaches and the results are predictive of severe irAE.

Accordingly, methods of the invention use a computational liquid biopsy framework to facilitate more precise and personalized cancer care including immunotherapy response and toxicity prediction.

In certain embodiments, the invention employs the insight that clonally-diverse activated CD4 memory T cells, and more specifically CXCR5-PD1hi peripheral helper T (Tph) cells, specifically underpin ICI-mediated toxicity in melanoma patients. To address this hypothesis, we perform CyTOF, scRNA-seq, scV(D)J-seq and immunoSEQ to broadly assess T and B cell states in pre- and on-treatment blood to (1) determine whether Tph levels in pretreatment blood are predictive of severe irAE development in melanoma patients treated with combination immunotherapy and to determine whether Tph clonotypes preferentially expand on-treatment during combination immunotherapy in patients who develop severe toxicity.

While the prediction of severe irAEs from peripheral blood is important clinically, patients who experience some degree of toxicity have also been shown to have better durable immunotherapy response rates. Therefore, it may be challenging to make clinical decisions regarding immunotherapy without also considering the probability of durable response. The present invention alleviates these challenges using cell-free DNA methylation sequencing to predict 1) immunotherapy toxicity and 2) durable immunotherapy response concurrently from pre-treatment plasma using both cell-state signatures and an agnostic machine learning approach, which are validated in held-out cohorts (Aim 3). By doing so, the present invention provides tools used to lay the foundation for future clinical trials where immunotherapy decision-making is guided by the risk versus benefit of combination immunotherapy using the liquid biopsy biomarkers defined here.

Severe, life-threatening or fatal immune-related adverse events (irAEs) occur in ˜60% of melanoma patients treated with combination immune checkpoint inhibitors (ICIs). While recent data suggest that autoreactive lymphocytes play a key role in facilitating irAEs, the pathophysiology underlying severe irAE development remains unclear. Due to this lack of knowledge, there is no way in clinical practice to predict who develop these severe toxicities, leading to unacceptably high rates of treatment-related morbidity and mortality. Additionally, no minimally invasive clinical assay can simultaneously predict the risk of irAE development and the benefit of immunotherapy response pre-treatment, making treatment personalization challenging. According to one aspect of the invention, CXCR5-PD1hi peripheral helper CD4 T cells (Tph) are a key determinant of future toxicity in melanoma patients receiving ICIs; are functionally linked to irAE development and are applicable to both toxicity and early response assessment through cell-free DNA methylation profiling. Clonally diverse activated CD4 memory T cells are significantly elevated in pretreatment blood from melanoma patients who develop severe or life-threatening irAEs following anti-PD1 and anti-CTLA4 combination therapy (Nature Medicine, 2022). In data from 13 melanoma patients, it is found that these same T cells have a gene expression profile enriched in Tph cells, a circulating T cell state elevated in autoimmune disorders such as rheumatoid arthritis, lupus and type 1 diabetes, but not yet rigorously evaluated in human irAE development.

Additionally, in data of plasma cell-free DNA (cfDNA) methylation profiles from 21 melanoma patients, striking signatures of immunotherapy response and toxicity were observed, with the latter mirroring activated CD4 memory T cells in the blood.

Here, peripheral blood from 100 melanoma patients was treated with dual anti-PD1/anti-CTLA4 therapy to determine whether Tph cells underlie severe irAE development at baseline, are associated with clonal expansion on-treatment, and can be leveraged, along with tissue-based signatures, to determine ICI response and toxicity from baseline cfDNA. Two innovative approaches underpin this. First, was the present inventors' development of a key enabling platform for benchmarking Tph that is predictive of irAE development organ-system-agnostically in a graded fashion from pre-treatment blood (Nature Medicine, 20226). Prior studies predicted irAEs from a single organ system, generalized to multiple organs poorly (AUC<0.68), or did not distinguish life-threatening grade 4 irAEs. Second, using a machine teaming method for profiling cellular ecosystems at scale, an ecosystem of 7 cell states was identified, which is more predictive of ICI response than >100 competing measures, including specialized biomarkers, and is detectable from cfDNA. Previous ICI response biomarkers were limited to modest cohort sizes, single genes, bulk signatures, or direct tissue biopsies.

Tph cells specifically underlie severe irAEs (not response) in melanoma patients treated with combination ICIs. To address this, mass cytometry was performed on pretreatment PBMCs acquired prospectively from 100 melanoma patients treated with combination ICIs, and scRNA-seq/scV(D)Jseq on 50 of these samples. Cell state frequencies were compared with irAE incidence and response.

Activated CD4 memory T cell clonotypes, and specifically Tph cells, preferentially clonally expand prior to severe irAE development. This may provide mechanistic insight, as any T cell state that preferentially expands prior to irAE onset is likely linked to irAE development. Accordingly, paired scRNA-seq/scV(D)J-seq was performed early on-treatment (cycle 2 day 1) from the same 50 patients profiled pretreatment by scRNA-seq in Aim 1. To validate these findings, immunoSEQ was performed on the same T cell states from pre- and early on-treatment blood from the remaining 50 melanoma patients in Aim 1.

Based on data from 21 patients, it was hypothesized that signatures of ICI toxicity and response can be inferred from pre-treatment cfDNA methylation profiles. Accordingly, NEBNext Enzymatic Methylseq was applied to pre-treatment cfDNA from the same 100 patients in Aim 1. After splitting into training/validation cohorts, published gene signatures of toxicity and response, refined signatures of toxicity and agnostic machine learning strategies to identify predictive signatures were assessed.

This study revealed irAE biology and new technology to improve melanoma outcomes. Further, the study validated that methylation profiles of cfDNA from ATMs is useful to accurately predict treatment response and/or toxicity.

Severe and debilitating toxicities, known as immune-related adverse events (irAEs), are a common side effect of many types of possible therapies for myriad conditions. This is especially true of therapies using dual immune checkpoint inhibition. Immune checkpoint inhibition is a paradigm-shifting treatment modality that has revolutionized outcomes for patients with melanoma, for which 100,000 new cases are diagnosed in the US each year. A standard-of-care for patients with metastatic melanoma is dual anti-PD1/anti-CTLA4 blockade (hereafter referred to as combination ICIs), which has better response and improved survival rates compared to single-agent ICIs. Unfortunately, combination ICIs can cause severe and potentially life-threatening irAEs (grade 3+) in up to 60% of melanoma patients treated with ICIs. This leads to early termination of anti-cancer treatment, hospitalization, intensive care unit admission, and even death. ICI-induced irAEs affect a variety of organ systems including lungs, heart, joints, thyroid, pituitary, liver, colon, nervous system, and skin. There is no clinical assay to determine pretreatment which patients experience severe irAEs, and the mechanisms underlying their development are poorly understood.

Life-threatening (grade 4) and fatal (grade 5) irAEs occur at a rate of 11%1 and 1.2%, respectively, in patients treated with dual ICIs, and at a rate of 5%1 and 0.4%, respectively, in patients receiving anti-PD1 monotherapy. Fatal irAEs typically arise early after the start of immunotherapy, at a median of just 14.5 days and 40 days after combination ICI and anti-PD1 monotherapy initiation, respectively. Unfortunately, no clinical assay can determine who experience life-threatening or fatal (grade 4-5) irAEs. Indeed, delineating the biological underpinnings of grade 4+irAEs could help clinicians select alternative therapeutic measures (i.e., withholding adjuvant immunotherapy in stage III melanoma after surgical resection, or favoring anti-PD1 treatment, which has a lower severe irAE rate than combination ICIs) in patients at risk of dying from ICIs.

Several groups have investigated potential biomarkers of ICI-induced toxicity based on blood or tumor analysis. However, those studies have generally focused on early on-treatment prediction or single organ systems, with only modest pretreatment performance independent of organ system and without demonstrated ability to distinguish multiple irAE grades. More recently a serum antibody approach was utilized to predict severe ICI-induced irAEs pretreatment in melanoma. However, those signatures differed significantly based on the immunotherapy regimen employed and there was no evidence that multiple irAE grades could be discriminated. Thus, prior studies have not revealed a common immunological state that precedes irAE development with ability to predict irAE severity independent of organ system and ICI regimen.

It has been shown herein that clonally diverse activated CD4 T effector memory cells are strongly associated with irAE onset and severity, with implications for pinpointing the cellular underpinnings of irAE development.

Levels of clonally diverse activated CD4 T effector memory cells are significantly higher in pre-treatment peripheral blood in melanoma patients who developed severe toxicity from immunotherapy. Those cells also preferentially expanded on-treatment in an exploratory cohort of patients who developed severe toxicity after combination ICI treatment. Clonally diverse activated CD4 T effector memory cell abundance remained a significant pre-treatment predictor of the onset and severity of toxicity independent of organ system involvement.

A major barrier to improving melanoma outcomes is insufficient ability to jointly determine the probability of both ICI response and irAE development prior to treatment. While the prediction of immunotherapy-induced toxicities is critical and could help prevent substantial morbidity, or even mortality, it has been shown that patients who develop toxicity are more likely to respond to treatment. Thus, it may be challenging to make important clinical decisions using an assay that only determines the probability of toxicity without also considering response, as it complicates the risk-benefit calculation. Liquid biopsies of cell-free DNA (cfDNA) are a proven approach for minimally invasive tumor profiling and can now be applied to interrogate cell states in tumors and blood that are predictive of clinical outcomes.

Cell-free DNA, which is continuously shed into blood plasma, is a useful analyte for measuring diverse physiological and pathological states. While tumor-derived cfDNA has been shown to decrease from pre- to on-treatment in durable responders to ICIs, most cancer-focused cfDNA assays measure somatic mutations, limiting their scope to cancer cells. In contrast, epigenomic alterations, such as methylation levels, can be used to detect cell-type-of-origin from cfDNA, making them generally applicable to cell state profiling. While no pre-treatment cfDNA assay has been shown to predict both ICI response and risk of irAEs, our new pilot data demonstrate proof-of-concept ability to address this challenge.

As a proof-of-concept, the presently-disclosed methods are used to determine whether CXCR5-PD1hi peripheral helper CD4 T cells (Tph)—a key subset of CD4 memory T cells linked to diverse manifestations of autoimmune disease-underlie severe irAE development, are associated with irAE-related clonal expansion, and can be leveraged, along with tissue-based signatures, to forecast ICI response and toxicity from pretreatment cfDNA. Tph cells were identified as a likely phenotype underlying the association between activated CD4 memory T cells and irAE development. A proof-of-principle study has also been performed using whole-genome cfDNA methylation profiling from pretreatment plasma of 21 melanoma patients and have identified novel cfDNA signatures of ICI response and irAE development.

Successful completion of this advances the understanding of the cell states that underlie irAEs, which could facilitate therapeutic targeting of this population in the future to prevent or abrogate ICI-induced irAEs without sacrificing efficacy and facilitates clinical translation via a cfDNA assay that enables both ICI toxicity and response prediction.

The scientific premises underlying this study include the following. First, clonally diverse activated CD4 memory T cells in pretreatment peripheral blood are associated with irAE development, independent of organ system, in patients with melanoma receiving ICI therapy. Second, CD4 memory T cells consist of diverse phenotypic subsets, including Tph cells, which are linked to several autoimmune disorders such as rheumatoid arthritis, lupus, and type 1 diabetes, but have not yet been rigorously evaluated in human irAE development. Third, delineating the T cell phenotypic state(s) that preferentially expand(s) on-treatment in relation to irAE development can nominate the phenotypic state(s) underlying irAE development. Fourth, minimally invasive cfDNA methylation profiling can be used to interrogate the composition of tumor and blood-derived cell states from peripheral blood plasma.

In aspects of methods of the invention, the use of a circulating T cell biomarker to predict risk of severe irAEs from pretreatment blood is taught. Previously reported features associated with irAE development show modest performance for predicting irAEs from pretreatment samples (AUC<0.68); were only described for single organ systems; lack clear mechanistic insight (e.g., autoantibodies); or were not shown to predict irAE grade. A biomarker combining activated CD4 memory T cell abundance and T cell receptor diversity can predict severe irAEs with high accuracy from pretreatment blood of melanoma patients (AUC of 0.90 in patients treated with combination ICIs), is independent of organ system, is biologically interpretable with mechanistic implications that form the basis for this proposal, and can also distinguish irAE grades, including life-threatening grade 4 irAEs from non-life-threatening but severe grade 3 irAEs.

In cancer, complex ecosystems of interacting cell types form powerful signaling networks that shape tumorigenesis. While single-cell genomics, spatial transcriptomics, and multiplexed imaging obtain high-resolution portraits of tumor cellular ecosystems, practical considerations have limited these assays in their scale, scope, and depth. To overcome these limitations, a machine learning framework for large-scale identification of cell states and ecosystems from single-cell, bulk, and spatially resolved expression data was developed. The machine learning framework identified a novel cellular ecosystem that is localized to the tumor core in carcinomas and melanoma; is comprised of seven lymphoid and myeloid states; is more strongly correlated with ICI response than 121 competing measures, including dedicated biomarkers; and is powerfully associated with response to combination ICIs when measured in a pilot study of 21 pretreatment cfDNA methylation profiles from melanoma patients.

Single-cell profiling of pretreatment blood reveals two T cell features—activated CD4 memory T cells and T cell receptor (TCR) diversity—associated with severe irAE development in patients with melanoma. In a recently published study, high dimensional, single-cell profiling was applied to a discovery cohort of pretreatment blood samples from 18 patients with advanced melanoma, of which eight experienced severe irAEs after ICI initiation.

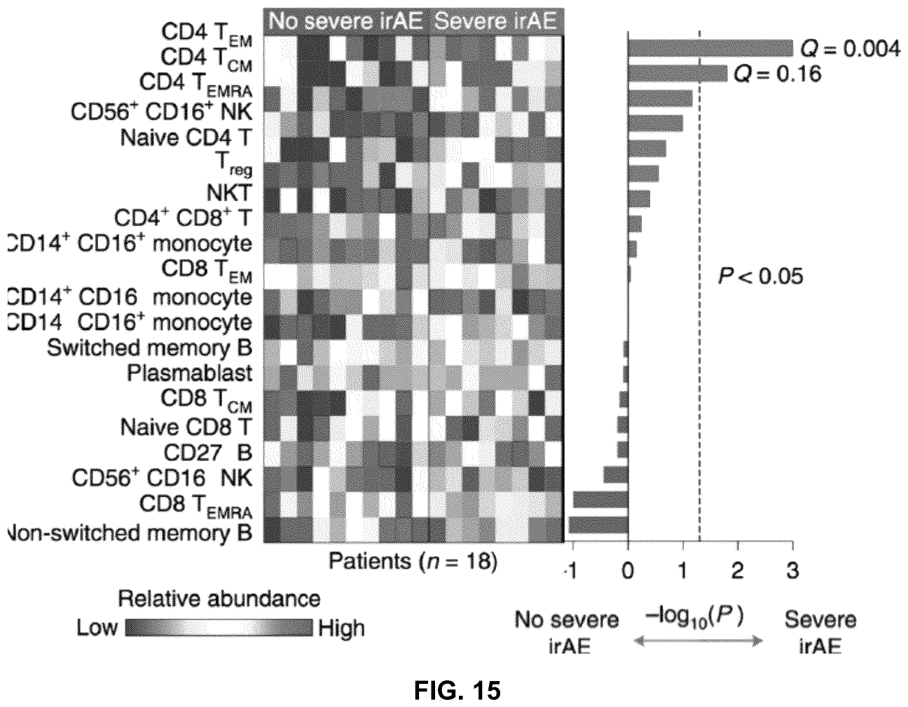

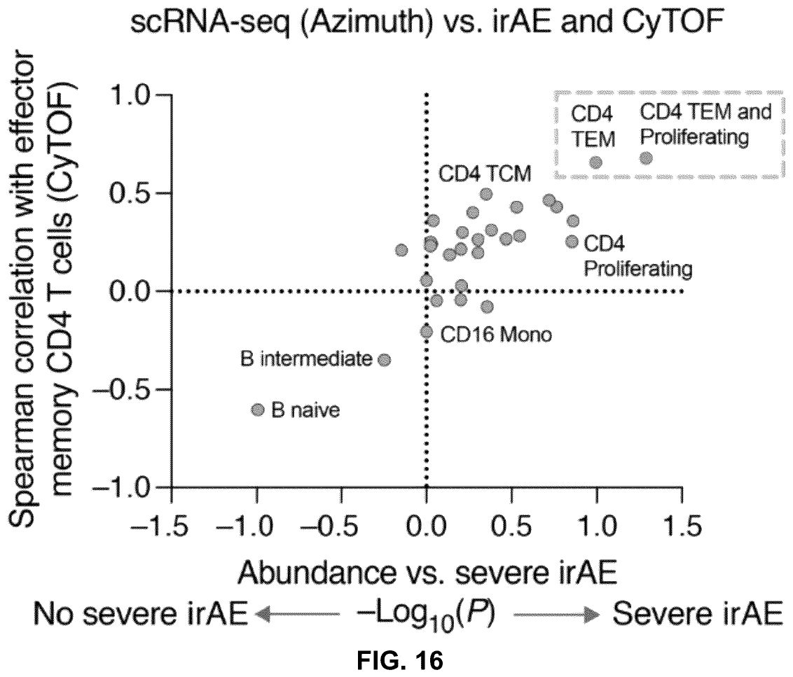

FIG. 15 and FIG. 16 give analysis of pretreatment peripheral blood for cellular determinants of severe irAE.

FIG. 15 shows relative abundance of 20 cell states by CyTOF in 18 patients, and association with irAE development. FIG. 15 shows By CyTOF, we found that elevated levels of CD4 effector memory T cells (TEM) cells were significantly associated with severe irAE development.

FIG. 16 shows cell state abundances (scRNA-seq) versus future irAE status and CD4 TEM cell frequency (CyTOF) in the same patients. FIG. 16 corroborates those findings by scRNA-seq and shows that CD4 TEM cells expressing activation markers are more strongly associated with severe irAEs. Given these results, it was hypothesized whether pretreatment TCR diversity might also correlate with severe ICI-induced irAEs.

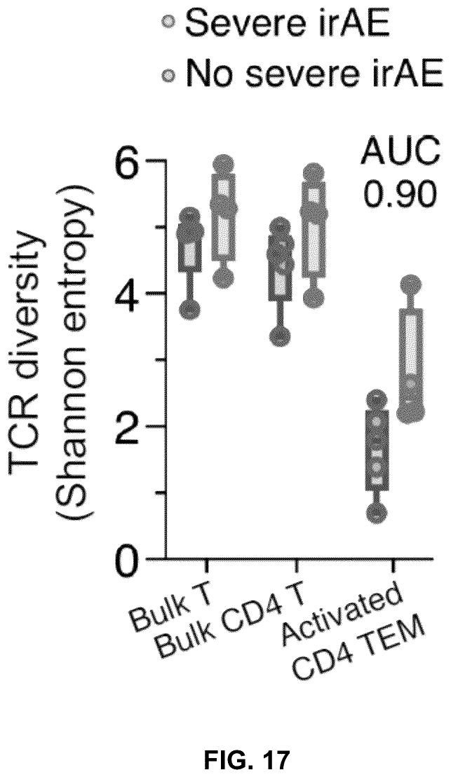

FIG. 17 shows that single-cell TCR clonotype diversity (Shannon entropy) of activated CD4 TEM cells was elevated in patients who experienced severe irAEs (AUC=0.90, P=0.05). This association was driven by TCR richness—or the number of unique clonotypes relative to total PBMCs. While this association was diminished or absent in other T cell subsets, when combining all evaluable T cells, a striking trend between bulk TCR diversity and severe irAE development was observed, driven by CD4 TEM and TEM-like T cells (AUC=0.80). These findings suggest that a more diverse TCR repertoire at baseline in CD4 TEM cells, broadly reflected in bulk peripheral blood, is associated with the development of severe irAEs. FIG. 17. TCR diversity by scV(D)J-seq. Pretreatment TCR clonotypic diversity in activated CD4 TEMs, bulk CD4 T cells and bulk T cells, stratified by irAE status.

Bulk RNA-seq profiling of pretreatment blood confirms the association between activated CD4 memory T cell levels, TCR diversity, and severe irAE development.

Using bulk RNA sequencing to analyze pretreatment blood from 53 additional melanoma patients treated with ICIs and split into two bulk cohorts (I and 2), we queried immune composition using CIBERSORTx66 and TCR clonotype diversity using MiXCR67.

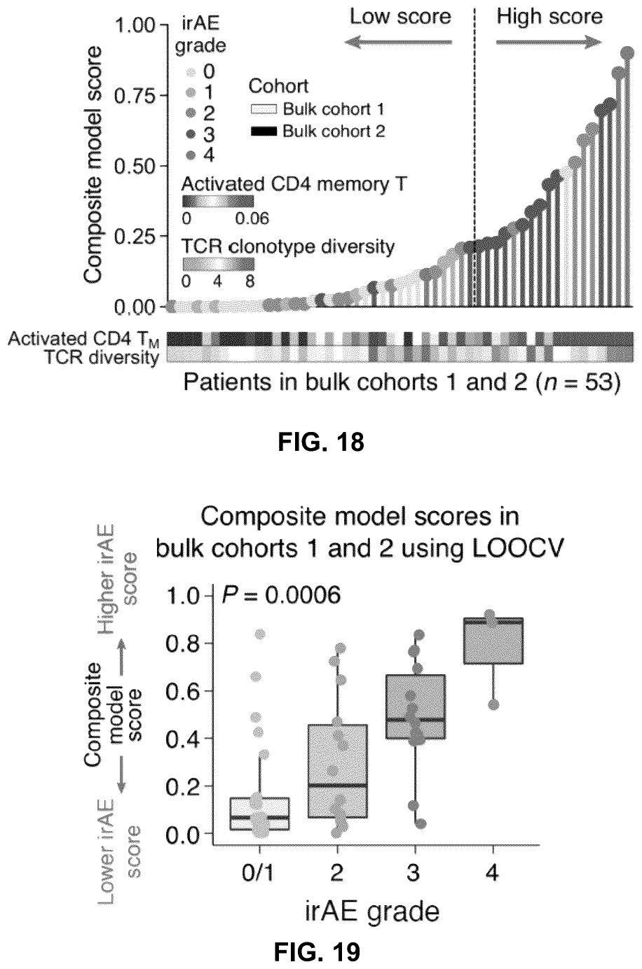

FIG. 18 shows that among 13 cell states queried by CIBERSORTx, only activated CD4 memory T cell levels were associated with severe irAE development. Moreover, higher TCR clonotype diversity in bulk blood predicted severe irAE development across organ systems.

Next, we used integrative modeling for prediction of irAE risk and grade from pretreatment blood. It was hypothesized whether a model integrating activated CD4 memory T cell abundance and TCR diversity from bulk RNA-seq data might outperform either feature alone.

FIG. 18 shows pretreatment activated CD4 memory T cell levels and TCR clonotype diversity vs. irAE development in 53 patients.

FIG. 19 shows Association between composite model and highest irAE grade.

FIG. 19 shows that using a logistic regression framework to train a bivariable model, the resulting pretreatment model was highly predictive for severe irAE development across organ systems (AUC=0.90, P=0.0004), including for patients treated with anti-CTLA-4/anti-PD1 combination therapy (AUC=1.0, P=0.04) and for distinguishing different irAE grades (FIG. 19). The model remains predictive for severe irAE development across clinical and epidemiologic subgroups and is not significantly associated with durable clinical benefit, emphasizing its specificity for irAE biology. To test whether the pretreatment model could predict time to severe irAE, patients were assigned to high versus low groups by defining an optimal cut-point in bulk cohort 1. In held-out bulk cohort 2, patients in the high group experienced severe irAEs within a median of 1.74 months after treatment initiation, whereas the vast majority of patients in the low group never experienced a severe irAE (P<0.0001, hazard ratio [HR]=11.6).

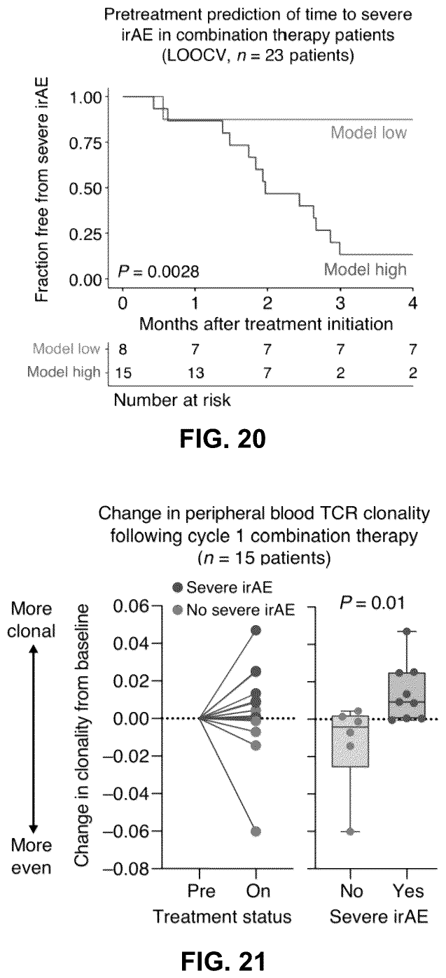

FIG. 20 shows time to severe irAE in combination ICI patients, stratified by composite model score.

FIG. 20 shows that Similar results were seen for each ICI type separately, whether assessed in bulk cohort 2 (P<0.025, HR=8.3 and 14.8 for combination and PD1, respectively) or across cohorts by leave-one-out cross-validation (LOOCV) (P=0.0028 and HR=12.2 for combination therapy, FIG. 20; P=0.03 and HR=9.0 for PD1 therapy). This candidate biomarker also predicted time-to-severe irAE in multivariable models independently of ICI type, age, sex, and other clinical parameters (P=0.005, Z=2.81).

TCR clonal expansion following ICI initiation is correlated with severe irAE development and points to a role for CD4 TEMs in irAE etiology. Here immunoSEQ was used to profile bulk TCR-ß repertoires in paired pre- and early on-treatment PBMC samples collected from 15 melanoma patients treated with combination ICIs. FIG. 21 shows TCR clonal dynamics in relation to severe irAE development in combination ICI patients. FIG. 21 shows that increased TCR clonal expansion was preferentially associated with severe irAEs. We also perform scRNA-seq and scTCR-seq in 3 of these severe irAE patients, in which preferential expansion of the activated CD4 TEM compartment was observed among clones detected in both blood draws. Extending this analysis to a larger cohort, as outlined in Aim 2, further elucidates whether a specific T cell state preferentially expands upstream of clinical irAE development.

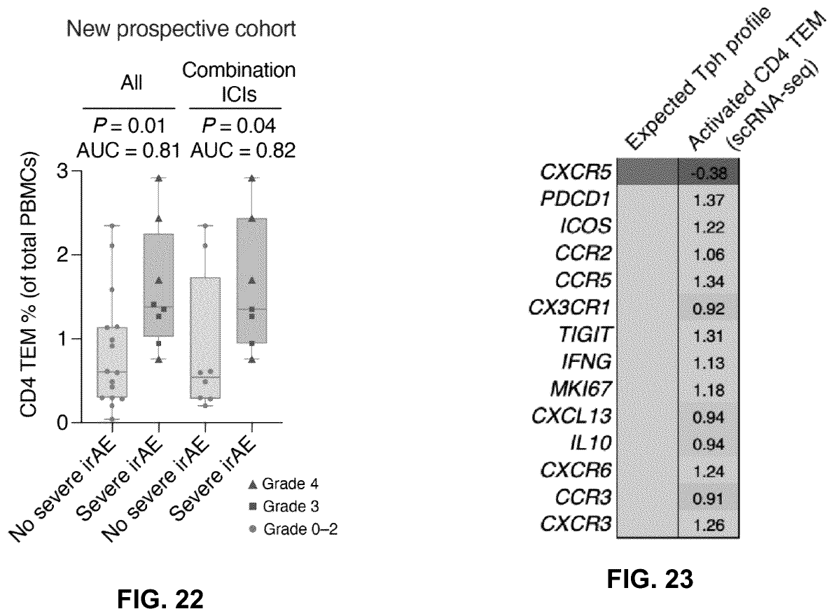

As a first step toward validating the findings described herein, 24 new melanoma patients were recruited from Yale and Washington University, and blood was collected on cycle 1 day 1, with 63% treated with combination ICIs and the rest with anti-PD1 monotherapy. Among these patients, 8 developed severe irAEs (including 4 with life-threatening irAEs) spanning 9 organ systems and 16 did not. Patients, including those treated with combination ICIs, who did not develop severe irAE had low circulating CD4 TEMs measured by CyTOF, comparable to healthy controls. FIG. 22 shows a Preliminary prospective validation and association of pretreatment Tph levels with severe irAE status. FIG. 22 shows CD4 TEM levels measured by CyTOF, stratified by ICI regimen and irAE status. FIG. 23 shows CD4 TEM expression profile (mean log 2 fold change vs. other CD4 subsets) from our published scRNA-seq data, compared to expected Tph profile.

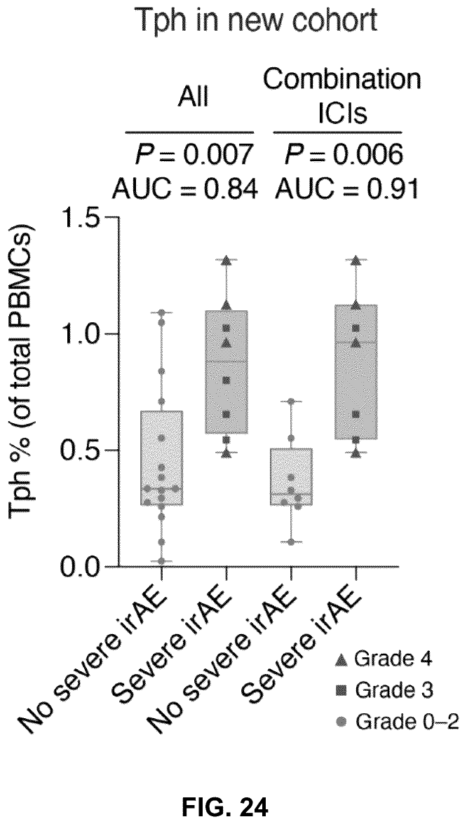

FIG. 24 shows Tph levels measured by CyTOF and stratified similar to A. Group comparisons performed with a two-sided Wilcoxon test. FIG. 22 shows that patients who developed severe irAEs had significantly higher CD4 TEM levels. Indeed, when splitting the cohort into two groups based on the median CD4 TEM level, patients with low CD4 TEMs had significantly better freedom from severe irAE than those with high CD4 TEMs (P=0.013; HR=6.7), corroborating.

CXCR5-PD1 hi Tph cells are a pathogenic CD4 T cell state that has been implicated in several autoimmune disorders including rheumatoid arthritis7, lupus8, type 1 diabetes, IgA nephropathy, and IG4-related diseases. Using scRNA-seq data of pretreatment blood from a recent study, activated CD4 TEM cells were examined, which correlate with severe irAE development. FIG. 23 shows that activated CD4 TEM cells that correlate with severe irAE development with a remarkably similar expression profile to Tph cells. As such, it was hypothesized whether profiling Tph cells would enhance irAE predictability over quantification of CD4 TEM levels alone. While Tph cells were not evaluable in the published CyTOF cohort owing to the absence of required markers, new CyTOF panel was designed to finely delineate these cells.

Elevated Tph cell levels in baseline blood are strongly predictive of severe irAEs in pilot data. Within CD4 memory T cells, Tph markers (CXCR5-PD1hi) were highly enriched by CyTOF. FIG. 24 shows that Moreover, Tph levels in pretreatment CD4 memory T cells were more strongly correlated with severe irAE development than CD4 TEMs, with potential to discriminate grade 4 from 3 irAEs.

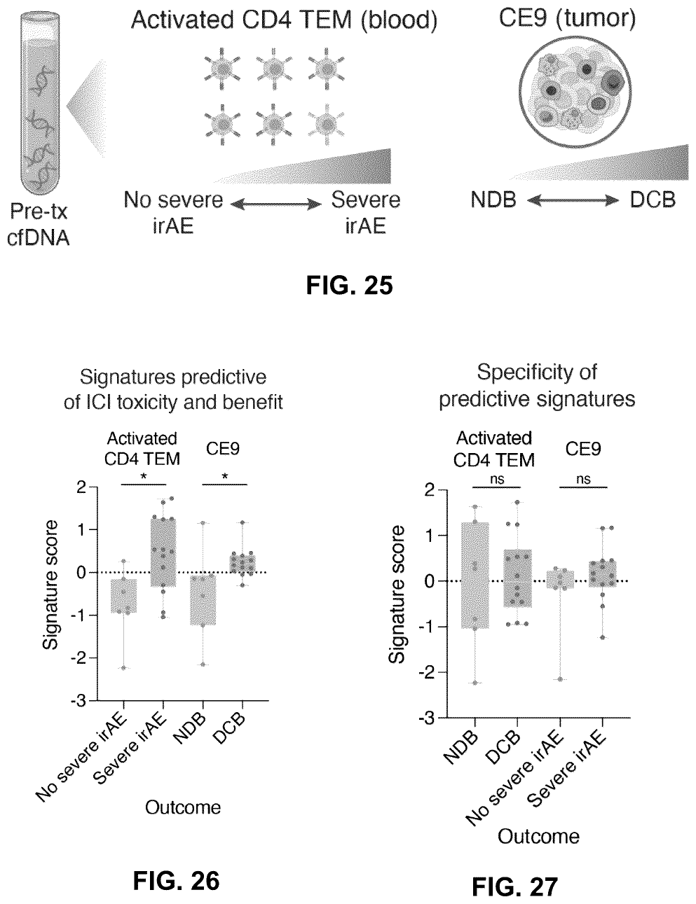

Pretreatment cfDNA methylation profiles have promise for predicting ICI toxicity and response. To determine whether future ICI toxicity and ICI are detectable from baseline cfDNA, we extracted pre-ICI cfDNA from plasma samples of 21 patients from our published retrospective melanoma cohort treated with combination ICIs. FIG. 25 shows a pretreatment cell-free DNA analysis to simultaneously predict response and toxicity. FIG. 25 gives a schema showing cell-free DNA derived from activated CD4 TEMs (for irAE prediction) and from the tumor microenvironment (CE9; for response prediction). FIG. 26 shows the activated CD4 TEM score and CE9 score correlated with toxicity and response status, respectively.

FIG. 27 gives an Inverted outcomes analysis (compared to FIG. 26) to query biomarker specificity. *P<0.05, two-sided Wilcoxon test. FIG. 25 shows a whole genome Enzymatic Methyl (EM)-seq performed to a median coverage of 30′ and analyzed promoter methylation levels of genes associated with ICI toxicity and response. For the former, the top 20 signature genes of CD4 TEM cells profiled by scRNA-seq from a recent study were assessed.

FIG. 26 shows that patients treated with combination ICIs who experienced severe irAEs had a significantly elevated CD4 TEM score from this promoter-level methylation analysis compared to those who experienced no severe irAE (AUC=0.84). Moreover, patients who experienced durable clinical benefit to combination immune checkpoint blockade had a significantly higher CE9 score derived from plasma cell-free DNA compared to non-responders (AUC=0.84). FIG. 27 shows that indicative of specificity, CE9 was not predictive of toxicity, and the CD4 TEM score was not associated with immunotherapy response. These data suggest that pre-treatment cell-free DNA methylation analysis can be used to concurrently predict both immunotherapy response and toxicity by specifically querying the corresponding cell states evident in plasma cell-free DNA from tumor tissue and blood, respectively.

These data support that pretreatment blood samples from melanoma patients who develop severe irAEs are generally enriched in activated CD4 memory T cells, including Tph cells, the latter of which may underpin irAE development. These results are validated and refined in a larger cohort of melanoma patients, and blood and tissue-based signatures queried in cfDNA reliably forecast ICI toxicity and response in new patients.

The data above indicate: (i) clonally diverse activated CD4 TEM cells are enriched pretreatment in the peripheral blood of melanoma patients at risk for severe toxicity from immune checkpoint blockade; (ii) activated CD4 TEM cells appear to undergo preferential clonal expansion on-treatment in melanoma patients treated with combination ICIs who develop severe irAEs; (iii) CXCR5-PD1hi Tph cells outperform CD4 TEM cells for predicting severe irAE development in a newly accrued melanoma cohort, with potential to discriminate severe from life-threatening irAEs; and (iv) cell-free DNA methylation profiling is a promising approach to concurrently predict immunotherapy toxicity and response from pretreatment plasma.

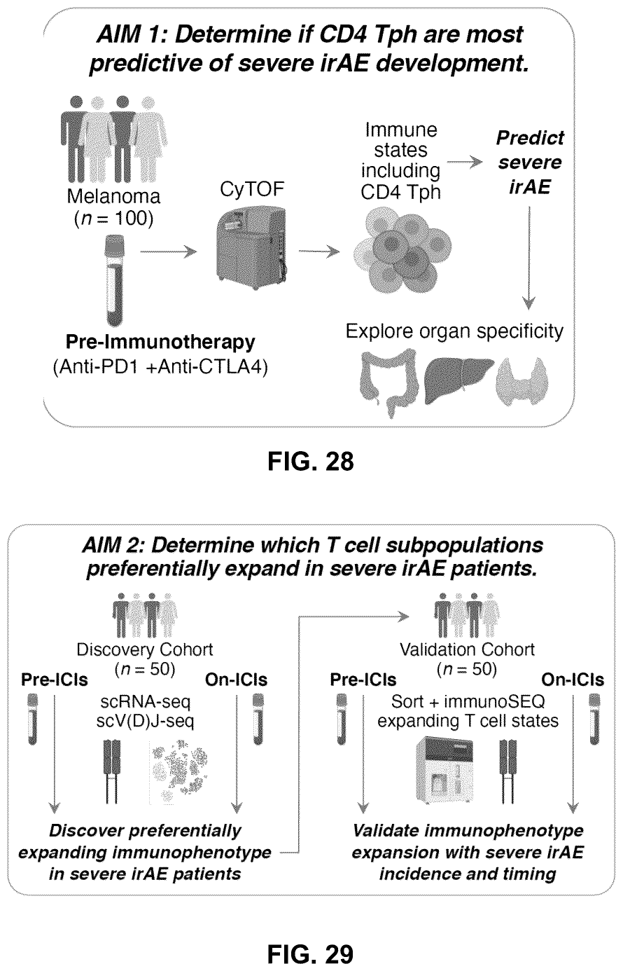

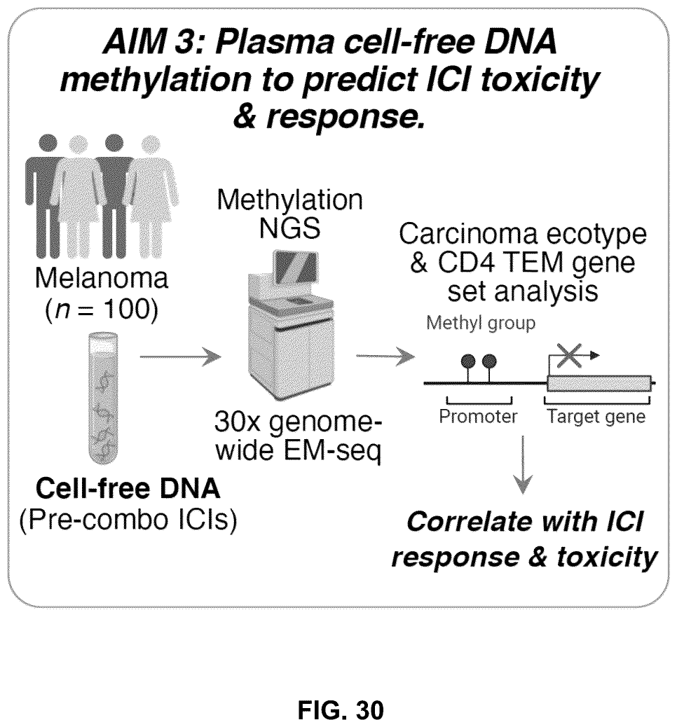

It is determined whether pretreatment CD4 Tph cells in circulation are associated with irAE risk (FIG. 28), whether they preferentially expand on-treatment in irAE patients (FIG. 29), and whether a liquid biopsy assay has utility for simultaneous assessment of ICI toxicity and response (FIG. 30).

FIG. 28 is a schematic of the illustrating in Melanoma (n=100) to determine if CD4 Tph are most predictive of severe irAE development.

FIG. 29 is a schematic illustrating use of the invention to determine which T cell subpopulations preferentially expand in severe irAE patients. FIG. 30 is a schematic illustrating the use of Plasma cell-free DNA methylation to predict ICI toxicity & response.

A multi-institutional cohort of 100 patients with advanced-stage unresectable melanoma treated with combination ICIs (anti-PD1/anti-CTLA4)(standard-of-care, see Section A.1) were recruited from Yale Cancer Center and Washington University Siteman Cancer Center, and peripheral blood collected before and early during ICI treatment. In a first study, droplet-based scRNA-seq and time-of-flight mass cytometry was performed on all major pretreatment PBMC populations to determine whether baseline levels of Tph cells are most predictive of severe irAE development. Next, paired scRNA-seq/scV(D)J-seq of pre and early on-treatment blood was performed along with immunoSEQ TCR sequencing to determine whether Tph (or another T cell population) preferentially expand(s) on-treatment in patients experiencing severe irAEs. Finally, it was determined whether pretreatment cell state-derived signatures or agnostic machine learning can effectively predict ICI toxicity and response using a novel cfDNA methylation assay. All results were correlated with incidence, grade, and timing of irAEs.

Peripheral blood samples from melanoma patients were collected from Yale Cancer Center and Washington University Siteman Cancer Center. Thus, peripheral blood samples are readily available for all three aims of this study within the first 1-2 years with sufficient follow-up for the proposed analyses. PBMC and plasma samples are collected pre-treatment (cycle 1 day 1) and on-treatment (cycle 2 day 1) from all patients. Clinical data collection includes age, sex, race, histologic subtype, disease stage, irAE severity (CTCAE v5), irAE timing, irAE-afflicted organ system(s), durable clinical response, number of ICI cycles, overall survival, progression-free survival, and institution. It was determined whether Tph cell levels in pretreatment blood are predictive of severe irAE development in melanoma patients treated with anti-PD1/anti-CTLA4 therapy.

The diversity and predictive power of irAE-associated factors across melanoma patients has important implications for the breadth of diagnostics and therapies that would be needed to maximize ICI efficacy. Based on preliminary data, it was hypothesized that elevated baseline levels of Tph cells specifically underlie severe irAEs (not response) in melanoma patients treated with combination ICIs. An alternative hypothesis is that Tph cells do not specifically associate with irAEs and that other cell subsets, including the parent CD4 memory T cell population, may be more predictive. To address this, mass cytometry is performed on pretreatment PBMCs acquired prospectively from 100 melanoma patients treated with combination ICIs, and scRNA-seq/scV(D)J-seq on 50 of these samples. Cell state frequencies are compared with irAE incidence and response to determine which hypothesis is correct.

Several risk groups are important for melanoma pathogenesis. For example, melanoma can be clinically subdivided into cutaneous, mucosal, and ocular subtypes, with ocular melanoma generally having a worse prognosis and lower response rates to immunotherapy. Separately, melanoma is ˜20 times more common in whites than in blacks, and metastatic melanoma is more common in men than in women. Also, studies have shown that immunotherapy response rates are higher in patients who experience irAEs, especially those involving certain organ systems (i.e. skin). Extensive clinical data be collected on each subject, and we consider known covariates (e.g., age, race, histologic subtype, durable response status) within this aim, as well as sites of organ tissue toxicity.

Peripheral blood was collected from 100 melanoma patients treated with combination ICIs (anti-PD1/anti-CTLA4). Peripheral blood is collected prior to the initiation of combination ICIs in melanoma patients (on cycle 1 day 1) in ˜2 K2EDTA Vacutainer tubes (˜20 mL)(Becton Dickinson) and processed within 1 hour of phlebotomy. An expected yield is ˜20 million PBMCs from ˜20 mL of blood. PBMCs are isolated using Lymphoprep (Stem Cell Technologies) per the manufacturer's instructions, resuspended in freezing media (90% FBS/10% DMSO), and cryopreserved at −80° C. in 10% dimethylsulfoxide/90% fetal bovine serum for 24 hours in a Mr. Frosty container (Nalgene), then stored in liquid nitrogen until further cellular processing.

Cryopreserved pre-treatment PBMC samples are thawed by holding cryovials in a 37° C. water bath for 1-2 minutes without submerging the cap. Subsequently, ˜3×106 PBMCs in single-cell suspension be incubated in Human TruStain FcX (BioLegend) at room temperature for 10 minutes to block nonspecific antibody binding, followed by incubation per the manufacturer's instructions with Cell-ID Cisplatin (Fluidigm) along with 50 metal-conjugated antibodies against cell surface and intracellular molecules (Fluidigm) including 38 that were recently reported on plus additional markers for central and effector memory T cells (i. e., CD62L, CCR7, CD27), T cell exhaustion (i. e., PD1, TIGIT) and CD4 T cell subsets (i. e., Th1, Th2, Th17, T peripheral helper [Tph] including CXCR5-PD1hi Tph cells) as we utilized to generate recent pilot data (FIG. 24). This may help determine whether activated CD4 TEMs predicting severe toxicity are preferentially CD4 Th1/2/17/ph.

Cells are then washed and stained with Cell-ID Intercalator-IR (Fluidigm), diluted in PBS containing 1.6% paraformaldehyde (Electron Microscopy Sciences) and stored at 4° C. until acquisition. After a wash step, sample acquisition is performed using the Helios System (Fluidigm) at an event rate of <400.

To reduce technical variation between sample, Ce beads were used in each sample and the output files normalized together using Bead Normalizer v0.3. To further minimize technical variability, sample processing and acquisition batches are limited, the same reagent lots used across all samples, and no major adjustments to Helios calibration settings between sample runs.

CyTOF data are analyzed with Cytobank v9.4 (Beckman Coulter) using the FlowSOM algorithm for hierarchical cluster optimization and the viSNE algorithm for visualization of high-dimensional data75,76. Cell subpopulation identification and data visualization be performed by manual gating with canonical markers using Cytobank v9.4.

For the first 50 patients accrued to the prospective cohort, single-cell RNA and single-cell V(D)J sequencing on pre-treatment PBMCs is performed. Single-cell suspensions from PBMC samples are obtained as above and prepared to a concentration of ˜1,000 viable cells per uL using a hemacytometer (Thermo Fisher Scientific) for cell counting, according to the manufacturer's instructions. Single-cell suspensions subsequently undergo library preparation for scRNA-seq with paired scV(D)J-seq using the 5′ transcriptome kit (10× Genomics) according to the manufacturer's instructions. Complementary DNA libraries at concentrations targeting 5,000 cells per sample be sequenced on a NovaSeq instrument (Illumina) with 2×100 base pair paired-end reads targeting 20,000 read pairs per cell.

Raw scRNA-seq reads are barcode-deduplicated and aligned to the hg38 reference genome using Cell Ranger v3.1.0, yielding sparse digital count matrices, which are analyzed to identify cell types and states using Seurat v4+77. Outlier cells are identified and removed based on the following criteria: (1)>25% mitochondrial content or (2) cells with <100 or >1,500-3,000 expressed genes depending on sample-level distributions. After normalization (NormalizeData) and variable feature identification (FindVariableFeatures), we applied FindlntegrationAnchors to identify anchors and IntegrateData to perform batch correction. Principal component analysis (PCA) is applied and uniform manifold approximation and projection (UMAP) using the most variable genes and top principal components. FindClusters is applied to identify cell types and states, which are assigned to major cell lineages based on the expression of canonical marker genes, and separately using a reference-guided annotation framework within Seurat v4 (Azimuth78) to project the scRNA-seq dataset onto a PBMC atlas of 161,764 cells spanning 6 major lineages and 27 finer-grain subsets.

Raw scV(D)J-seq reads were mapped with Cell Ranger v7.0 to the reference refdata-cellranger-vdj-GRCh38-altensembl-4.0.0 with the resulting clonotype assemblies downloaded from the Loupe V(D)J browser v3.0.0 (10× Genomics). To calculate T cell receptor (TCR) diversity, Shannon entropy (R package vegan v.2.5-379) relative to total PBMCs is used for each T cell subset. B cell receptor (BCR) clonotypes are similarly analyzed across B cell subsets for IGK, IGL and IGH chains.

All identified immune cell states were related across CyTOF, scRNAseq and scV(D)J-seq data including subset-specific TCR/BCR Shannon entropy with the incidence of symptomatic irAE (grade 2+), severe irAE (grade 3+) and life-threatening irAE (grade 4+) development, including by receiver operating characteristic area under the curve (ROC AUC) analysis, Kaplan-Meier analysis, Cox proportional hazards regression, Wilcoxon rank-sum test when comparing two groups, and Kruskal-Wallis test when comparing >2 groups simultaneously. Identified T/immune cell states are correlated with irAE development in a grade-by-grade fashion using the Jonckheere-Terpstra test for ordered data and also correlate circulating immunophenotypic state abundances with organ-specific toxicities in an exploratory fashion (i.e., colitis, myocarditis, pneumonitis, hepatitis, thyroiditis, hypophysitis, etc.). Multivariable models be used to verify independence from other clinical indices. Multiple hypothesis correction be performed as appropriate using the Benjamini-Hochberg method.

Pooling retrospective cohort and new prospective analysis (Section C. 5) for patients receiving combination ICIs and profiled by CyTOF (n=30), the association between pretreatment levels of CD4 TEM cells and severe irAE development has an effect size of 1.07. Based on this, at least 22 patients per group are needed to achieve α=0.01 and 1−β=0.8. For Tph cells, which were only evaluable in a new prospective cohort, the effect size was 2.05 for discriminating severe irAEs from combination ICIs, requiring 7 patients per group (alpha=0.01, 80% power). A cohort of 100 patients satisfy these requirements, as it is expected to include ˜60 of 100 patients with severe irAEs. The ability to identify cell populations from scRNA-seq data depends on three key variables: the number of cells profiled, the fold change between population-specific genes, and the number of population-specific genes. It is anticipated that at least 1,000 cells are needed to detect rare populations down to 1% (10 cells) with an effect size of >0.89 for identifying individual differentially expressed genes (alpha=0.05, 80% power). As we anticipate sequencing approximately 250k total cells (˜5k per sample for 50 samples), we expect to be well-powered to detect modest effect sizes.

The data, show (i) validation of the previously-published result that CD4 effector memory T cells are enriched in pretreatment peripheral blood of melanoma patients who develop severe irAEs6, (ii) that CXCR5-PD1hi CD4 Tph cells are the CD4 memory T cell state most predictive of severe irAE development and that their baseline levels correlate with toxicity severity in a graded fashion, (iii) that Tph cells enriched pre-treatment in patients who experience toxicity also have elevated TCR diversity, and (iv) whether additional immunophenotypic cell states are associated with organ-specific toxicities.

The heterogeneity of potential mechanisms underlying ICI-induced irAEs has complicated the development of therapeutic strategies to mitigate or avoid them. However, any T cell state that preferentially expands on-treatment prior to irAE onset is likely linked, either directly or indirectly, to irAE etiology. In pilot analyses of 15 melanoma patients treated with combination ICIs, we identify on-treatment clonal expansion of bulk T cells associated with severe irAEs. In three patients for whom we performed joint scRNAseq and scV(D)J-seq, we identify a shift toward clonally expanding activated CD4 TEM cells, but not CD8 T cells, in on-treatment blood preceding severe irAE development6. Based on our preliminary data, we hypothesize that activated CD4 effector memory T cell clonotypes, and specifically Tph cells, preferentially expand on-treatment prior to severe irAE development. Our alternative hypothesis is that alternative T/B cell subsets (or no T/B cell subsets) preferentially expand on-treatment prior to severe irAE development.

For the first 50 patients accrued to the prospective cohort, we perform single-cell RNA and single-cell V(D)J sequencing on the early on-treatment PBMCs (in addition to the pre-treatment PBMCs profiled in Aim 1) using the same protocol described in Section D. 4. d. 4.

Paired pre- and on-treatment scRNA-seq and scV(D)J-seq data be analyzed as described in Sections D.4.d.5 and D.4.d.6 and in our previous work. To maximize stringency and avoid classification artifacts, when annotating CD4 and CD8 T cell-derived clonotypes, we initially only consider TCR clonotypes with uniform expression of positive lineage markers (CD4>0 and CD8A/B=0 for CD4 T cells; CD8A or CD8B>0 and CD4=0 for CD8 T cells). In previous work, 69% of all clonotypes could be unambiguously labeled by this approach.

For each T cell state identified in in both paired pre- and on-treatment scRNA-seq data, we evaluate TCR-b repertoire richness and evenness using Pielou's evenness, which is robust to the number of clones per sample, with increased 1—evenness associated with increased clonality. Clonal expansion for each T cell state then be inferred by analyzing the difference in clonality between on- and pre-treatment timepoints. We also analyze individual clonotypes private to pre and on-treatment samples as well as persistent clones-defined as productive TCR-ß CDR3 nucleotide sequences shared between paired pre- and on-treatment blood samples. For the latter, differences in productive frequencies between paired samples be analyzed on a per-clonotype basis, thereby allowing assessment of clonotype dynamics, both individually and in aggregate, to determine whether persistent clonotypes in any T cell state preferentially expand after ICI initiation, but prior to the development of severe irAEs. These analyses be repeated for B cell states.

Following confirmation (or identification) of a clonally expanding immunophenotype that is most significantly associated with severe irAE in 50 patients (D.5.c.4), we sort it from PBMCs collected pre- and early on-treatment from the remaining 50 patients in the cohort. Specifically, ˜5 million PBMCs were treated with TruStain FcX Fc receptor blocking solution (BioLegend) for 10 minutes at room temperature, then stained with fluorophore-tagged surface antibodies specific to the T/B cell state of interest for 30 minutes at room temperature, and then sorted with operator assistance using a Sony SY3200 Synergy instrument at the Siteman Flow Cytometry Core at WashU following exclusion of DAPIpositive cells and putative doublets based on forward and side scatter analysis. Confirmation of sort performance be performed by analyzing the flow cytometry output using FlowJo v10.

Here, immunoSEQ TCR-b chain profiling, or BCR, was performed on paired pre- and early on-treatment sorted PBMCs. First, genomic DNA is extracted from each sorted population using the DNeasy Blood & Tissue kit (Qiagen) and submitted for survey-resolution immunoSEQ (Adaptive Biotechnologies). Data from productive rearrangements be exported using the immunoSEQ Analyzer online tool and evaluated for repertoire richness and diversity using Pielou's evenness. Pre-treatment-normalized clonality for the sorted cell state then be compared across irAE grades to confirm the predicted phenotype.

Additionally, time-to severe irAE development was assessed on the basis of degree of immunophenotypic clonal expansion, which can further implicate a functional connection. Kaplan-Meier analysis was used after dividing into tertiles and also perform multivariable Cox regression while accounting for the time between blood draws and other covariates.

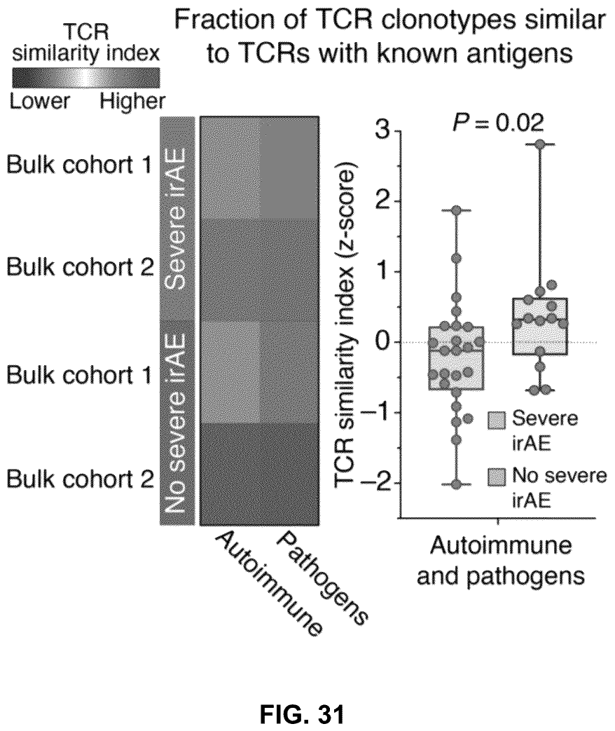

Expanding TCR clonotypes in peripheral blood might show a greater propensity to recognize self- and disease-associated antigens in patients destined to develop severe irAEs. FIG. 31 compares the similarity of expanding TCR clonotypes to an external database of CDR3 sequences with known antigens in autoimmunity, cancer, and pathogenic infection (McPAS-TCR). A similarity index per clonotype and per patient be determined as the sum of the percent TCR matches over edit distances 1, 2 and 3, and compared between patients based on severe irAE status. FIG. 31 shows TCR clonotypes from Lozano et al. compared to the McPAS-TCR database of CDR3 sequences with known antigen specificity, stratified by antigen type and irAE status. Wilcoxon test was used for group comparison.

The association between TCR clonal expansion (1−Pielou's evenness) and severe irAE onset in patients treated with combination ICIs has an effect size of 1. Based on this, 15 patients per group (with and without severe irAE) are needed to achieve α=0.01 and 1−β=0.8. We anticipate an even higher effect size for this association if we can successfully isolate the T cell state driving this signal. Thus, training/validation cohorts of 50 patients each (including ˜30 with severe and ˜20 without severe irAEs) are expected to be sufficiently powered for the proposed study.

We hypothesize that signatures of ICI toxicity and response are detectable in pretreatment cfDNA methylation profiles and can reliably predict corresponding clinical outcomes in melanoma. Consistent with this, data from 21 patients demonstrate that promoter methylation profiling of pretreatment cfDNA can forecast ICI toxicity and benefit using published signatures (FIG. 25-FIG. 27). First, we apply NEBNext Enzymatic Methyl-seq to pre-treatment cell-free DNA from the same 100 patients described above. After splitting into training/validation cohorts, we assess our published gene signatures of toxicity and response, refined signatures of toxicity and agnostic machine learning strategies to identify predictive signatures.

Briefly, about 20 mL of blood was collected in K2EDTA Vacutainer tubes (Becton Dickinson) at 1,200 g for 10 minutes, separate out plasma from PBMCs, and then spin the plasma again at 1,800 g to remove any remnant of PBMCs before storing the double-spun plasma in ˜2 mL aliquots at ˜80° C.

The cfDNA is extracted from plasma using the QiaAmp Circulating Nucleic Acid kit (Qiagen) according to the manufacturer's instructions. cfDNA concentration is measured with a Qubit 4.0 Fluorometer using the dsDNA High Sensitivity assay kit (Thermo Fisher Scientific) with fragment size assessed by Agilent 2100 Bioanalyzer with the High Sensitivity DNA kit (Agilent Technologies). A median of 50 ng is inputted into EM-Seq library preparation per the manufacturer's instructions. Libraries are sequenced on a NovaSeq 6000 (Illumina) targeting 30× genome-wide coverage.

Following alignment and determination of methylated sites using Bismark with default parameters, promoter methylation levels were analyzed (1 kb upstream of the gene body) in gene signatures associated with ICI toxicity. The top 10, 20, 50 and 100 signature genes were examined (log 2 fold change) from activated CD4 memory T cells profiled by scRNA-seq, and the cell state most associated with severe irAE development was identified. These analyses were performed looking at the 1 kb promoter regions as well as looking across the full gene and the promoter region.

For each gene set, signature scores—defined as 1 minus the mean promoter methylation level—are adjusted against the background of each sample by randomly sampling the same number of genes from the whole transcriptome and calculating 1 minus the mean promoter methylation level. The latter is performed ten times, then averaged and subtracted from the original signature score. Adjusted signature scores were compared from different gene set/region permutations with the incidence of severe irAE development in the first 50 patients accrued to the cohort (Discovery), and train discriminative cutpoints by applying Youden's J statistic to receiver-operating characteristic (ROC) analyses. Using CpGs from the top 100 signature genes (selected as above), we also train a series of machine learning models (SVM, logistic regression, random forest) as comparators. All predictors are assessed by LOOCV to identify the leading approach.

As validation, the best performing predictor to was compared to identically processed cfDNA EM-seq data from the remaining 50 patients as well as data from our pilot study and assess performance for predicting severe irAE by ROC analysis, two-sided Wilcoxon test, and Kaplan-Meier analysis for freedom from severe toxicity analyzed by log-rank test, as well as multivariable Cox proportional hazards models for freedom from severe toxicity. We also assess the graded association of these EM-seq-derived gene signatures with irAE grade by plotting their values against irAE grade, with statistical testing performed via the non-parametric Jonckheere-Terpstra test for ordered data.

Next cell-state-specific genes were interrogated comprising the cellular ecosystems that we previously identified in tumor tissue, including CE9, a proinflammatory ecosystem strongly predictive of immunotherapy response. Promoter methylation levels were analyzed in plasma cell-free DNA using the data analysis pipeline described above but against durable clinical benefit (DCB) to immunotherapy (defined as no progression by RECIST 1.1 criteria on standard of care imaging for ≥6 months after ICI start). Gene sets be defined as the top 10, 20, 50, and 100 signature genes per cell state comprising each ecosystem.

Using the same sequencing alignment and methylation analysis protocol as above, an agnostic machine learning system was used to identify patients at risk for severe irAE and those likely to achieve durable clinical benefit from combination ICIs. The algorithm also addresses predictive ability to discriminate irAE grade. Given considerations of computational efficiency, as an initial feature selection step, we identify all CpGs with significant differences by two-sided Wilcoxon test between 1) patients who developed severe irAE versus those who did not, and 2) patients who achieved durable clinical benefit (DCB) versus those with no durable benefit (NDB). CpGs with nominal significance (P<0.05) then are used to train XGBoost, an extreme gradient-boosted decision tree model, to distinguish defined outcomes, first by LOOCV to optimize decision tree parameters in our initial 50-patient discovery cohort, and then in held-out Validation Cohorts 1 and 2 (D. 6. c. 3). Performance is compared to the results of Sections above using ROC AUC analysis for binary variables and PFS/OS using Kaplan-Meier analysis and multivariable Cox proportional hazards regression.

In the cfDNA data (FIG. 25-FIG. 27), the association between an activated CD4 TEM signature and severe irAE onset in patients treated with combination ICIs had an effect size of 1.43. The association between ecosystem signatures and DCB had an effect size of 0.9. Based on this, 20 patients per clinical group are needed to achieve α=0.05 and 1−β=0.8. With ˜60% expected severe irAE rate and ˜60% DCB rate, training/validation cohorts of 50 patients each are sufficiently powered.

The data show that (i) signatures of activated CD4 memory T cells, and more specifically CD4 Tph cells, in genome-wide methylation profiles of pretreatment cfDNA can predict severe irAE development in discovery and validation cohorts, and (ii) that signatures obtained using machine learning algorithms can be applied to pretreatment cfDNA methylation data to predict durable response and survival in discovery and validation cohorts. We also determine whether (iii) machine learning can outperform (i) and (ii) in predicting ICI toxicity and benefit from the same data.

Methods of the invention were applied to combinatorial genomic and epigenomic analysis of cfDNA in high-risk, castration-resistant prostate cancer to reveal prognostic liquid biopsy signatures. The LiquidTME technology can be applied to risk-stratify patients with locally advanced or metastatic cancer—Distinguish patients with molecularly lower risk disease from those with molecularly higher-risk disease. In this way, seamlessly risk-stratify across cancer types to help clinicians modulate/strengthen/personalize treatment regimens.

What is claimed is:

1. A method of predicting a disease outcome in a subject, the method comprising:
identifying epigenetic modifications in sequence data, the epigenetic modifications including methylation status, fragment size, and/or end motifs incell-free nucleic acid obtained from a liquid biopsy sample;
identifying an aberrant tissue microenvironment and assigning a tissue of origin to the cell-free nucleic acid based on the epigenetic modifications;
determining cell states in the aberrant tissue microenvironment using patterns of epigenetic modifications; and
predicting the outcome of a disease in the subject based on the cell states in the aberrant tissue microenvironment.

2. The method of claim 1, wherein the aberrant tissue microenvironment is selected from a tumor microenvironment (TME), an inflammatory microenvironment (IME), a tissue transplant microenvironment (TTME), and a pathogen microenvironment (PME).

3. The method of claim 2, wherein the circulating cell-free nucleic acids are from microenvironment infiltrating lymphocytes.

4. The method of claim 3, wherein the disease is cancer and the aberrant tumor microenvironment is a TME.

5. The method of claim 4, wherein predicting the outcome of cancer in the subject includes one or more of diagnosing the subject with a cancer, predicting remission, predicting recurrence, assessing minimal residual disease, predicting response to an immunotherapy, and predicting immunotherapy toxicity.

6. The method of claim 5, wherein the subject has a low measured tumor mutational burden.

7. The method of claim 4, wherein predicting the outcome of cancer in the subject further comprises determining that the cell state in the TME has surpassed the pre-malignant threshold.

8. The method of claim 4, wherein determining cell states comprises measuring T cell abundance and/or diversity from patterns in the epigenetic modifications.

9. The method of claim 8, wherein the measured T cell diversity is a T cell receptor (TCR) diversity of a patient's immune repertoire.

10. The method of claim 8, further comprising inferring an abundance of T cell effector memory cells from the patterns.

11. The method of claim 10, further comprising predicting a risk of an adverse event in response to, or non-response to, immunotherapy wherein the measured TCR diversity and/or inferred T cell effector memory cell abundance is beneath a predetermined threshold.

12. The method of claim 10, further comprising predicting a positive outcome in response to an immunotherapy wherein the measured TCR diversity and/or inferred T cell effector memory cell abundance is above a predetermined threshold.

13. The method of claim 1, wherein the step of determining cell states comprises sequencing the nucleic acid to produce the sequence data that include methylated bases; mapping the sequence data to a reference to identify promotors of a plurality of genes; and identifying the cell states based on sets of the genes having methylated promotors.

14. The method of claim 1, wherein identified epigenetic modifications include promoter methylation.

15. The method of claim 1, further comprising querying a cell state atlas for the patterns in the epigenetic modifications.

16. The method of claim 15, further comprising providing a profile describing cell states of a plurality of cells in an aberrant tissue microenvironment in a patient.

17. The method of claim 4, further comprising providing a profile of tumor-infiltrating leukocytes in a tumor microenvironment based on the patterns in the epigenetic modifications.

18. The method of claim 4, wherein the cell-free nucleic acids comprise nucleic acids from non-tumor cells and wherein the step of determining cell states in the aberrant tissue microenvironment comprises generating a profile of the TME.

19. The method of claim 18, wherein the non-tumor cells comprise one or more of somatic cells, immune cells, and/or cells from the tumor margin.

20. The method of claim 19, wherein the profile of the TME includes one or more epigenetic pattern correlated with tumor progression and/or somatic regression.

21. The method of claim 20, further comprising predicting the toxicity of an immunotherapy in the subject using the profile of the TME.

22. The method of claim 21, wherein predicting toxicity of the immunotherapy is based on a level of epigenetic modification of promotors in genes involved in a predetermined T cell transcriptional state; and
predicting immunotherapy response based on a level of promotor methylation in the genes in a predetermined tumor ecotype.

23. The method of claim 22, wherein the predetermined T cell transcriptional state is specific to CD4 T cells.

24. The method of claim 22, wherein the predetermined tumor ecotype is carcinoma ecotype 9 or carcinoma ecotype 1.

25. The method of claim 21, wherein predicting toxicity of the immunotherapy is based on a level of epigenetic modification of promotors in genes involved in the expression of one or more cytokine; and
predicting immunotherapy response based on a level of promotor methylation in the genes in a predetermined tumor ecotype.

26. The method of claim 25, wherein the epigenetic modification is predictive of immunotherapy toxicity and is selected from one or more of:
promoter hypomethylation of interleukin-10;
promoter hypermethylation of interleukin-6; and
promoter hypermethylation of interleukin-7.
